(19) United States
(12) Patent Application Publication
(10) Pub. No.: US 2026/0117301 A1
(43) Pub. Date: Apr. 30, 2026

(54) REFERENCE STANDARDS FOR PLASMID DNA CONFORMATION ASSESSMENT AND SIZING ANALYSIS

(71) Applicant: Revvity Health Sciences, Inc., Waltham, MA (US)

(72) Inventors: Dipti Rasiklal Mehta, Waltham, MA (US); James White, Waltham, MA (US); Menel Ben Frej, Waltham, MA (US); Shreyas Shah, Waltham, MA (US); Lloyd Bwanali, Waltham, MA (US)

(73) Assignee: Revvity Health Sciences, Inc., Waltham, MA (US)

(21) Appl. No.: 19/371,498

(22) Filed: Oct. 28, 2025

Related U.S. Application Data
(60) Provisional application No. 63/712,867, filed on Oct. 28, 2024.

(51) Int. Cl. C12Q 1/6876; G01N 33/58

(52) U.S. Cl. CPC C12Q 1/6876; G01N 33/582

(57) ABSTRACT

Kits for assessing a proportion of supercoiled plasmid DNA compared to linear plasmid DNA, and/or a contaminant, in a fluid sample containing plasmid DNA according to aspects of the present disclosure include: a DNA reference standard including supercoiled pDNA and/or linear pDNA. Methods of assessing double-stranded plasmid DNA in a fluid sample according to aspects of the present disclosure include comparing characteristics of double-stranded plasmid DNA in a fluid sample to a reference standard representative of one or both of: supercoiled plasmid DNA and linear plasmid DNA, wherein the reference standard includes 1) at least two fragments of supercoiled pDNA of specified sizes and/or 2) at least two fragments of linear pDNA of specified sizes.

### REFERENCE TO RELATED APPLICATIONS

This application claims priority from U.S. Provisional Patent Application Ser. No. 63/712,867, filed Oct. 28, 2024, the entire content of which is incorporated herein by reference.

### FIELD OF THE INVENTION

The present disclosure relates generally to plasmid DNA (pDNA) conformation and sizing assessment. The present disclosure relates specifically to assessment of a desired pDNA in a sample presumed to include greater than 80% supercoiled isoform of the pDNA or greater than 80% linear isoform of the pDNA, or less than 5% of supercoiled isoform of the pDNA or less than 5% of linear isoform of the pDNA, to determine one or more of: presence, relative amount, and size of the desired supercoiled pDNA and/or the desired linear pDNA in the sample, and to detect impurities in the sample.

### BACKGROUND OF THE INVENTION

Plasmid DNA is an increasingly important delivery vector for gene therapy applications. Supercoiled pDNA is quite stable and has the highest efficiencies of both expression and transduction making supercoiled plasmid DNA the most desirable form of pDNA for use in applications such as DNA vaccines and gene therapy. Linear pDNA is also of interest in particular gene therapy applications, such as stable transformations. However, pDNA is subject to degradation during production and storage processes, resulting in open circular and/or linear forms of plasmid DNA which may be undesirable in some applications. The FDA has recommended a supercoiled plasmid DNA content of preferably greater than 80% (Guidance for Industry, Considerations for Plasmid DNA Vaccines for Infectious Disease Indications, November 2007, 11 pages, Page 3). However, standards and methods of their use for pDNA conformation assessment and/or size analysis are lacking. Thus, there is a continuing need for improved standards and methods of their use for pDNA conformation assessment and/or size analysis.

### SUMMARY OF THE INVENTION

Methods of assessing double-stranded plasmid DNA in a fluid sample to determine one or more of: conformation of the double-stranded plasmid DNA, size of the double-stranded plasmid DNA, and a proportion of supercoiled plasmid DNA compared to linear plasmid DNA according to the present disclosure include: contacting the double-stranded plasmid DNA of the fluid sample with a detectable label which preferentially labels double-stranded plasmid DNA thereby labeling double-stranded plasmid DNA, producing labeled double-stranded plasmid DNA; flowing the labeled double-stranded plasmid DNA through a polymeric separation medium in a microchannel into a detection region in fluid communication with the microchannel, the microchannel having a first end, a second end, and length extending between the first end and the second end, the detection region in signal communication with at least one sensor capable of detecting a signal from the detectable label of the labeled double-stranded plasmid DNA, whereby supercoiled double-stranded plasmid DNA and linear double-stranded plasmid DNA present in the labeled double-stranded plasmid DNA are detectably separated by flowing the labeled double-stranded plasmid DNA through the polymeric separation medium of the microchannel, thereby separating labeled supercoiled plasmid DNA from labeled linear plasmid DNA when both labeled supercoiled plasmid DNA and labeled linear plasmid are present; detecting the detectable label of the labeled double-stranded plasmid DNA in the detection region to determine: a) an amount of time taken by the labeled supercoiled plasmid DNA and/or labeled linear plasmid to flow through the polymeric separation medium in the microchannel into the detection region, indicative of a flow characteristic of the labeled supercoiled plasmid DNA and/or labeled linear plasmid in the fluid sample, and/or b) strength of the signal of the detectable label in the detection region, representative of an amount of supercoiled plasmid DNA and/or linear plasmid DNA, present; and comparing a) and/or b) to a reference standard representative of one or both of: supercoiled plasmid DNA and linear plasmid DNA, wherein the reference standard comprises 1) at least two fragments of supercoiled pDNA of specified sizes and/or 2) at least two fragments of linear pDNA of specified sizes and, based on the comparison, determining one or more of: conformation of the double-stranded plasmid DNA, size of the double-stranded plasmid DNA, and a proportion of supercoiled plasmid DNA compared to linear plasmid DNA in the fluid sample.

According to aspects of the present disclosure, the reference standard includes: 1) at least two fragments of supercoiled pDNA having a size in the range of about 2000 to about 20000 nucleotides in length, wherein the first of the at least two fragments of supercoiled pDNA is smaller than the second of the two fragments of supercoiled pDNA by at least about 1000 nucleotides in length, and/or 2) at least two fragments of linear pDNA in the range of about 2000 to about 20000 nucleotides, wherein the first of the two fragments of linear pDNA is smaller than the second of the two fragments of linear pDNA by at least about 1000 nucleotides in length.

According to aspects of the present disclosure, the reference standard includes: 1) at least three fragments of supercoiled pDNA of specified sizes and/or 2) at least three fragments of linear pDNA of specified sizes.

According to aspects of the present disclosure, the reference standard includes: 1) at least three fragments of supercoiled pDNA of specified sizes and 2) at least three fragments of linear pDNA of specified sizes.

According to aspects of the present disclosure, the reference standard includes: 1) at least three fragments of supercoiled pDNA in the range of about 2000 to about 20000 nucleotides in length, wherein the first of the at least three fragments of supercoiled pDNA is smaller than the second of the at least three fragments of supercoiled pDNA by at least about 1000 nucleotides in length, and the second of the three fragments of supercoiled pDNA is smaller than the third of the at least three fragments by at least about 1000 nucleotides in length; and/or 2) at least three fragments of linear pDNA in the range of about 2000 to about 20000 nucleotides, wherein the first of the three fragments of linear pDNA is smaller than the second of the at least three fragments of linear pDNA by at least about 1000 nucleotides in length, and the second of the at least three fragments of linear pDNA is smaller than the third of the at least three fragments of linear pDNA by at least about 1000 nucleotides in length.

According to aspects of the present disclosure, the reference standard includes: a) a first fragment of supercoiled pDNA having a size in the range of 1.0-3.5 kilobase pairs (kbp), b) a second fragment of supercoiled pDNA having a size in the range of 3.5-6.0 kbp, and c) a third fragment of supercoiled pDNA having a size in the range of 6.0-9.0 kbp, wherein the size of the first fragment of supercoiled pDNA is smaller than the second fragment of supercoiled pDNA by at least 1000 bp, and wherein the size of the second fragment of supercoiled pDNA is smaller than the third fragment of supercoiled pDNA by at least 1000 bp.

According to aspects of the present disclosure, the reference standard includes: d) a first fragment of linear pDNA having a size in the range of 0.2-5.0 kilobase pairs (kbp), e) a second fragment of linear pDNA having a size in the range of 3.0-7.0 kbp, and f) a third fragment of linear pDNA having a size in the range of 5.0-11.0 kbp, wherein the size of the first fragment of linear pDNA is smaller than the second fragment of linear pDNA by at least 1000 bp, and wherein the size of the second fragment of linear pDNA is smaller than the third fragment of linear pDNA by at least 1000 bp.

According to aspects of the present disclosure, the reference standard includes: a) a first fragment of supercoiled pDNA having a size in the range of 1.0-3.5 kilobase pairs (kbp), b) a second fragment of supercoiled pDNA having a size in the range of 2-6.0 kbp, and c) a third fragment of supercoiled pDNA having a size in the range of 6.0-10.0 kbp, wherein the size of the first fragment of supercoiled pDNA is smaller than the second fragment of supercoiled pDNA by at least 1000 bp, and wherein the size of the second fragment of supercoiled pDNA is smaller than the third fragment of supercoiled pDNA by at least 1000 bp; and the reference standard includes d) a first fragment of linear pDNA having a size in the range of 0.2-5.0 kilobase pairs (kbp), e) a second fragment of linear pDNA having a size in the range of 3.0-7.0 kbp, and f) a third fragment of linear pDNA having a size in the range of 5.0-11.0 kbp, wherein the size of the first fragment of linear pDNA is smaller than the second fragment of linear pDNA by at least 1000 bp, and wherein the size of the second fragment of linear pDNA is smaller than the third fragment of linear pDNA by at least 1000 bp.

According to aspects of the present disclosure, the concentration of the supercoiled pDNA and/or linear pDNA in the reference standard is in the range of 0.01 ng/μl to about 1 ng/μl for each of the fragments of supercoiled pDNA and/or linear pDNA of the reference standard.

According to aspects of the present disclosure, the detectable label of the double-stranded plasmid DNA in the fluid sample is an intercalator.

According to aspects of the present disclosure, the fragments of supercoiled pDNA and/or linear pDNA of the reference standard are labeled with a detectable label.

According to aspects of the present disclosure, the fragments of supercoiled pDNA and/or linear pDNA of the reference standard are labeled with a detectable label which is, or includes, an intercalator.

According to aspects of the present disclosure, the detectable label of the double-stranded plasmid DNA in the fluid sample is a first detectable label and the detectable label of the reference standard is a second detectable label, wherein the first detectable label and the second detectable label are intercalators, and wherein the emission maxima of the first and second intercalators are not detectably different.

According to aspects of the present disclosure, the detectable label of the double-stranded plasmid DNA in the fluid sample is a first detectable label and the detectable label of the reference standard is a second detectable label, wherein the first detectable label and the second detectable label are intercalators, and wherein the emission maxima of the first and second intercalators are detectably different.

According to aspects of the present disclosure, labeling the plasmid DNA comprises introducing the plasmid DNA into a well and/or microchannel of a microfluidic device, the well and/or microchannel comprising the polymeric separation medium and a detectable label which labels plasmid DNA isoforms producing labeled plasmid DNA isoforms in the well and/or microchannel.

According to aspects of the present disclosure, labeling the pDNA of the fluid sample and/or the pDNA of the reference standard, comprises introducing the pDNA of the fluid sample and/or the pDNA of the reference standard into a well and/or microchannel of a microfluidic device, together or separately, the well and/or microchannel comprising the polymeric separation medium and a detectable label which labels pDNA isoforms producing labeled plasmid DNA isoforms in the well and/or microchannel. According to aspects of the present disclosure, the detectable label of the pDNA of the fluid sample and/or the pDNA of the reference standard is an intercalator.

According to aspects of the present disclosure, the plasmid DNA has a size in the range of about 2000 to about 20000 nucleic acid base pairs in length.

According to aspects of the present disclosure, the plasmid DNA has a concentration in the range of about 0.02 ng/μl to about 2 ng/μl.

According to aspects of the present disclosure, flowing the labeled plasmid DNA through the polymeric separation medium includes application of a voltage gradient along the length of the microchannel between the first end and the second end, wherein the voltage gradient is in the range of about 500V to about 1500V.

According to aspects of the present disclosure, the labeled plasmid DNA is introduced into the well and/or microchannel by electrokinetic injection or pressure injection.

According to aspects of the present disclosure, the polymeric separation medium comprises a polymer selected from the group consisting of: about 0.08% to about 0.6% hydroxypropyl methylcellulose (HPMC) weight/weight (w/w) of the total weight of the polymeric separation medium having an average molecular weight in the range of about 80 kDa to about 120 kDa; about 0.08% to about 0.4% HPMC, having an average molecular weight in the range of about 90 kDa to about 160 kDa; about 0.08% to about 0.6% weight/weight (w/w) of the total weight of the polymeric separation medium hydroxyethyl cellulose (HEC) having an average molecular weight in the range of about 90 kDa to about 160 kDa; about 0.08% to about 0.6% weight/weight (w/w) of the total weight of the polymeric separation medium polyvinylpyrrolidone (PVP) having an average molecular weight in the range of about 90 kDa to about 130 kDa; about 0.05% to about 0.6% weight/weight (w/w) of the total weight of the polymeric separation medium polydimethylacrylamide (PDMA), having an average molecular weight in the range of about 80 kDa to about 500 kDa; about 5% weight/weight (w/w) of the total weight of the polymeric separation medium polyacrylamide gel; and non-cross-linked polyacrylamide.

According to aspects of the present disclosure, the polymeric separation medium comprises a buffer selected from the group consisting of: 1 mM-5 mM MgCl₂ (magnesium chloride); Tris Borate EDTA Buffer; HEPES (2-[4-(2-hydroxy ethyl) piperazin-1-yl]ethane sulfonic acid) with boric acid; Tris Buffer, and TAPS buffer (N-Tris(hydroxymethyl)methyl-3-aminopropanesulfonic acid).

According to aspects of the present disclosure, the polymeric separation medium comprises a stabilizer.

According to aspects of the present disclosure, the stabilizer is selected from the group consisting of: tris(hydroxymethyl)methylamino]propanesulfonic acid (TAPS), Tris/Borate/EDTA (TBE), and Tris-acetate-EDTA (TAE).

According to aspects of the present disclosure, the polymeric separation medium comprises: about 0.3% to about 0.6% polymer gel; and about 5% to about 20% TAPS buffer (N-Tris(hydroxymethyl)methyl-3-aminopropanesulfonic acid), and has a conductivity of about 1 ms/cm to about 3 ms/cm, and has a viscosity of about 0.25 cSt to about 1 cSt.

According to aspects of the present disclosure, the polymeric separation medium comprises: about 0.1% to about 0.6% polydimethylacrylamide (PDMA), having an average molecular weight in the range of about 80 kDa to about 800 kDa; and about 5% to about 20% TAPS buffer (N-Tris(hydroxymethyl)methyl-3-aminopropanesulfonic acid), has a conductivity of about 1 ms/cm to about 3 ms/cm, and has a viscosity of about 0.25 cSt to about 1 cSt.

According to aspects of the present disclosure, a migration time value for a reference standard of a given conformation; and based on alignment of the sample migration time to the standard migration time a sizing output is obtained.

Kits for assessing a proportion of supercoiled plasmid DNA compared to linear plasmid DNA, and/or a contaminant, in a fluid sample containing plasmid DNA according to aspects of the present disclosure include: a DNA reference standard including supercoiled pDNA and/or linear pDNA.

Kits for assessing a proportion of supercoiled plasmid DNA compared to linear plasmid DNA, and/or a contaminant, in a fluid sample containing plasmid DNA according to aspects of the present disclosure include: a DNA reference standard including: a) a first fragment of supercoiled pDNA having a size in the range of 1.0-3.5 kilobase pairs (kbp), b) a second fragment of supercoiled pDNA having a size in the range of 3.5-6.0 kbp, and c) a third fragment of supercoiled pDNA having a size in the range of 6.0-9.0 kbp, wherein the size of the first fragment of supercoiled pDNA is smaller than the second fragment of supercoiled pDNA by at least 1000 bp, and wherein the size of the second fragment of supercoiled pDNA is smaller than the third fragment of supercoiled pDNA by at least 1000 bp.

According to aspects of the present disclosure, the reference standard includes: d) a first fragment of linear pDNA having a size in the range of 0.2-5.0 kilobase pairs (kbp), e) a second fragment of linear pDNA having a size in the range of 3.0-7.0 kbp, and f) a third fragment of linear pDNA having a size in the range of 5.0-11.0 kbp, wherein the size of the first fragment of linear pDNA is smaller than the second fragment of linear pDNA by at least 1000 bp, and wherein the size of the second fragment of linear pDNA is smaller than the third fragment of linear pDNA by at least 1000 bp.

Kits for assessing a proportion of supercoiled plasmid DNA compared to linear plasmid DNA, and/or a contaminant, in a fluid sample containing plasmid DNA according to aspects of the present disclosure include: a DNA reference standard including: a) a first fragment of supercoiled pDNA having a size in the range of 1.0-3.5 kilobase pairs (kbp), b) a second fragment of supercoiled pDNA having a size in the range of 2-6.0 kbp, and c) a third fragment of supercoiled pDNA having a size in the range of 6.0-10.0 kbp, wherein the size of the first fragment of supercoiled pDNA is smaller than the second fragment of supercoiled pDNA by at least 1000 bp, and wherein the size of the second fragment of supercoiled pDNA is smaller than the third fragment of supercoiled pDNA by at least 1000 bp; and the reference standard includes d) a first fragment of linear pDNA having a size in the range of 0.2-4.0 kilobase pairs (kbp), e) a second fragment of linear pDNA having a size in the range of 3.0-7.0 kbp, and f) a third fragment of linear pDNA having a size in the range of 5.0-11.0 kbp, wherein the size of the first fragment of linear pDNA is smaller than the second fragment of linear pDNA by at least 1000 bp, and wherein the size of the second fragment of linear pDNA is smaller than the third fragment of linear pDNA by at least 1000 bp.

Kits for assessing a proportion of supercoiled plasmid DNA compared to linear plasmid DNA, and/or a contaminant, in a fluid sample containing plasmid DNA according to aspects of the present disclosure further include one or more of: one or more detectable nucleic acid labels which label double-stranded plasmid DNA, including sample plasmid DNA and/or reference standard fragments of plasmid DNA; a polymeric separation medium; a nucleic acid storage buffer; and a nucleic acid sample buffer.

Kits for assessing a proportion of supercoiled plasmid DNA compared to linear plasmid DNA, and/or a contaminant, in a fluid sample containing plasmid DNA according to aspects of the present disclosure further include one or more of: one or more detectable nucleic acid labels which label double-stranded plasmid DNA, including sample plasmid DNA and/or reference standard fragments of plasmid DNA, wherein the one or more detectable nucleic acid labels are fluorescent nucleic acid intercalators.

### BRIEF DESCRIPTION OF THE DRAWINGS

FIG. 1 is a diagram showing plasmid DNA (pDNA) isoforms including open circular plasmid DNA, linear plasmid DNA, and supercoiled plasmid DNA;

FIG. 2 graphically shows results in which the top line shows the reference standard; a separate peak for each of the three supercoiled pDNA fragments (“SC 2.6”, “SC 4.3”, “SC 6.7”) is observed along with a separate peak for each of the three linear pDNA fragments (“Lin 4000 bp”, “Lin 6000 bp”, “Lin 10000 bp”); the lower line shows three separate peaks for the supercoiled pDNA (“SC-4.3”), linear pDNA (Lin-4.3), and open circular pDNA (OC-4.3) determined by comparison to the reference standard trace above;

FIG. 3 graphically shows example data of isoform standard curves using a reference standard of the present disclosure; for each curve, the circled data points L1, L2 and L3 are measured isoform peaks from the ladder; the other data points are measured samples; the solid line represents peak to peak fitting from the reference ladder and the dashed trendline represents predicted sizing experimentally derived from the reference ladder;

FIG. 4 is a table showing an example of sizing accuracy using a reference standard of the present disclosure using the curves shown in FIG. 3;

FIG. 5 is a graph and table showing an example of impurity identification using a reference standard of the present disclosure wherein the results were obtained under similar conditions; and

FIG. 6 is a graph showing an example of impurity identification using a reference standard of the present disclosure.

### DETAILED DESCRIPTION

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; CRISPR/Cas: A Laboratory Manual, Doudna and Mali (eds), Cold Spring Harbor Laboratory Press, Cold Spring Harbor, NY, USA, 2016; D. L. Nelson and M. M. Cox, Lehninger Principles of Biochemistry, 4th Ed., W.H. Freeman & Company, 2004; J.-H. Fuhrhop et al. (Eds.), Organic Synthesis, Concepts and Methods, 3rd Ed., Wiley-VCH Verlag GmbH & Co. KGaA, 2003; Herdewijn, P. (Ed.), Oligonucleotide Synthesis: Methods and Applications, Methods in Molecular Biology, Humana Press, 2004; D. J. Taxman (ed.), siRNA Design, Methods and Protocols, Humana Press, 2012; Harlow, E. and Lane, D., Antibodies: A Laboratory Manual, Cold Spring Harbor Laboratory Press, 1988; J. D. Pound (Ed.) Immunochemical Protocols, Methods in Molecular Biology, Humana Press, 2nd ed., 1998; Chu, E. and Devita, V. T., Eds., Physicians' Cancer Chemotherapy Drug Manual, Jones & Bartlett Publishers, 2021; J. M. Kirkwood et al., Eds., Current Cancer Therapeutics, 4th Ed., Current Medicine Group, 2001; A Adejare (Ed.), Remington: The Science and Practice of Pharmacy, Elsevier, 23rd Ed., 2021; L. V. Allen, Jr. et al., Ansel's Pharmaceutical Dosage Forms and Drug Delivery Systems, 11th Ed., Wolters Kluwer, 2016; and L. Brunton et al., Goodman & Gilman's The Pharmacological Basis of Therapeutics, McGraw-Hill Education, 13th Ed., 2018.

The singular terms “a,” “an,” and “the” are not intended to be limiting and include plural referents unless explicitly stated otherwise or the context clearly indicates otherwise.

The terms “includes,” “comprises,” “including,” “comprising,” “has,” “having,” and grammatical variations thereof, when used in this specification, are not intended to be limiting, and specify the presence of stated features, elements, and/or components, but do not preclude the presence or addition of one or more other features, elements, components, and/or groups thereof.

The term “about” as used herein in reference to a number is used herein to include numbers which are greater, or less than, a stated or implied value by 1%, 5%, 10%, or 20%.

Particular combinations of features are recited in the claims and/or disclosed in the specification, and these combinations of features are not intended to limit the disclosure of various aspects. Combinations of such features not specifically recited in the claims and/or disclosed in the specification. Although each dependent claim listed below may directly depend on only one claim, the disclosure of various aspects includes each dependent claim in combination with every other claim in the claim set. As used herein, a phrase referring to “at least one of” a list of items refers to any combination of those items, including single members. As an example, “at least one of: a, b, or c” is intended to cover a alone; b alone; c alone, a and b, a, b, and c, b and c, a and c, as well as any combination with multiples of the same element, such as a and a; a, a, and a; a, a, and b; a, a, and c; a, b, and b; a, c, and c; and any other combination or ordering of a, b, and c).

The terms “first,” “second,” and the like are used herein to describe various features or elements, but these features or elements are not intended to be limited by these terms, but are only used to distinguish one feature or element from another feature or element. Thus, a first feature or element could be termed a second feature or element, and vice versa, without departing from the teachings of the present disclosure.

According to aspects of the present disclosure, reference standards and methods of their use for pDNA conformation assessment and/or pDNA size analysis are provided.

The term “conformation” as used herein refers to plasmid DNA isoforms including 1) linear plasmid DNA and 2) supercoiled plasmid DNA.

A reference standard according to aspects of the present disclosure includes 1) at least two fragments of supercoiled pDNA of specified sizes and/or 2) at least two fragments of linear pDNA of specified sizes.

A reference standard according to aspects of the present disclosure includes 1) at least two fragments of supercoiled pDNA in the range of about 2000 to about 20000 nucleotides in length, wherein the first of the two fragments of supercoiled pDNA is smaller than the second of the two fragments of supercoiled pDNA by at least about 1000 nucleotides in length, and/or 2) at least two fragments of linear pDNA in the range of about 2000 to about 20000 nucleotides, wherein the first of the two fragments of linear pDNA is smaller than the second of the two fragments of linear pDNA by at least about 1000 nucleotides in length.

A reference standard according to aspects of the present disclosure includes 1) at least three fragments of supercoiled pDNA of specified sizes and/or 2) at least three fragments of linear pDNA of specified sizes.

A reference standard according to aspects of the present disclosure includes 1) at least three fragments of supercoiled pDNA in the range of about 2000 to about 20000 nucleotides in length, wherein the first of the at least three fragments of supercoiled pDNA is smaller than the second of the at least three fragments of supercoiled pDNA by at least about 1000 nucleotides in length, and the second of the three fragments of supercoiled pDNA is smaller than the third of the at least three fragments by at least about 1000 nucleotides in length; and/or 2) at least three fragments of linear pDNA in the range of about 2000 to about 20000 nucleotides, wherein the first of the three fragments of linear pDNA is smaller than the second of the at least three fragments of linear pDNA by at least about 1000 nucleotides in length, and the second of the at least three fragments of linear pDNA is smaller than the third of the at least three fragments of linear pDNA by at least about 1000 nucleotides in length.

According to aspects of the present disclosure, a reference standard includes a) a first fragment of supercoiled pDNA having a size in the range of 1.0-3.5 kilobase pairs (kbp), b) a second fragment of supercoiled pDNA having a size in the range of 3.5-6.0 kbp, and c) a third fragment of supercoiled pDNA having a size in the range of 6.0-9.0 kbp, wherein the size of the first fragment of supercoiled pDNA is smaller than the second fragment of supercoiled pDNA by at least 1000 bp, and wherein the size of the second fragment of supercoiled pDNA is smaller than the third fragment of supercoiled pDNA by at least 1000 bp.

According to aspects of the present disclosure, a reference standard includes d) a first fragment of linear pDNA having a size in the range of 0.2-5.0 kilobase pairs (kbp), e) a second fragment of linear pDNA having a size in the range of 3.0-7.0 kbp, and f) a third fragment of linear pDNA having a size in the range of 5.0-11.0 kbp, wherein the size of the first fragment of linear pDNA is smaller than the second fragment of linear pDNA by at least 1000 bp, and wherein the size of the second fragment of linear pDNA is smaller than the third fragment of linear pDNA by at least 1000 bp.

According to aspects of the present disclosure, a reference standard includes a) a first fragment of supercoiled pDNA having a size in the range of 1.0-3.5 kilobase pairs (kbp), b) a second fragment of supercoiled pDNA having a size in the range of 2-6.0 kbp, and c) a third fragment of supercoiled pDNA having a size in the range of 6.0-10.0 kbp, wherein the size of the first fragment of supercoiled pDNA is smaller than the second fragment of supercoiled pDNA by at least 1000 bp, and wherein the size of the second fragment of supercoiled pDNA is smaller than the third fragment of supercoiled pDNA by at least 1000 bp; and the reference standard includes d) a first fragment of linear pDNA having a size in the range of 0.2-5.0 kilobase pairs (kbp), e) a second fragment of linear pDNA having a size in the range of 3.0-7.0 kbp, and f) a third fragment of linear pDNA having a size in the range of 5.0-11.0 kbp, wherein the size of the first fragment of linear pDNA is smaller than the second fragment of linear pDNA by at least 1000 bp, and wherein the size of the second fragment of linear pDNA is smaller than the third fragment of linear pDNA by at least 1000 bp.

As detailed further herein, the plasmid DNA in the fluid sample to be analyzed is generally present in a concentration in the range of about 0.02 ng/μl to about 10 ng/μl. According to aspects of the present disclosure, the plasmid DNA in the fluid sample to be analyzed is generally present in a concentration in the range of about 0.02 ng/μl to about 5 ng/μl. According to aspects of the present disclosure, the plasmid DNA in the fluid sample to be analyzed is generally present in a concentration in the range of about 0.02 ng/μl to about 2 ng/μl.

The concentration of the supercoiled pDNA in the reference standard is likewise in the range of 0.01 ng/μl to about 1 ng/μl for each of the at least two, or each of the at least three, fragments of supercoiled pDNA present. The concentration of the linear pDNA in the reference standard is likewise in the range of 0.01 ng/μl to about 1 ng/μl for each of the at least two, or each of the at least three, fragments of linear pDNA present. The concentration of each of the at least two or each of the at least three fragments of supercoiled or linear pDNA may vary independently, i.e., the concentrations do not have to be equal.

Fragments of supercoiled pDNA and fragments of linear pDNA included in a reference standard according to aspects of the present disclosure are generated by any of various methods, such as, for example, enzymatic digestion by one or more restriction enzymes, ligation of fragments of known size, or PCR amplification of known targets.

According to aspects of the present disclosure, methods are provided for determining the conformation and/or size and/or amount of a desired supercoiled pDNA and/or a desired linear pDNA in a fluid sample. Impurities in the fluid sample can also be detected. Such methods include contacting the pDNA in the fluid sample with a detectable label which preferentially labels double-stranded pDNA, thereby labeling linear pDNA and supercoiled pDNA in the fluid sample with the detectable label, producing labeled pDNA.

The labeled double-stranded plasmid DNA is flowed through a polymeric separation medium in a microchannel into a detection region in fluid communication with the microchannel, the microchannel having a first end, a second end, and length extending between the first end and the second end, the detection region in signal communication with at least one sensor capable of detecting a signal from the detectable label of the labeled double-stranded plasmid DNA, whereby supercoiled double-stranded plasmid DNA and linear double-stranded plasmid DNA, present in the labeled double-stranded plasmid DNA are detectably separated by flowing the labeled double-stranded plasmid DNA through the polymeric separation medium of the microchannel. Impurities which are not supercoiled double-stranded plasmid DNA or linear double-stranded plasmid DNA, such as multimers of the plasmid DNA or open circle form of the plasmid DNA are also detected.

The detectable label of the labeled double-stranded plasmid DNA is detected in the detection region to determine: a) an amount of time taken by the labeled plasmid DNA to flow through the polymeric separation medium in the microchannel into the detection region, indicative of a flow characteristic of the plasmid DNA isoforms in the fluid sample, and/or b) strength of the signal of the detectable label in the detection region, representative of the amount of supercoiled plasmid DNA and/or linear plasmid DNA present.

The detected amount of time and/or strength of signal is compared to a reference standard representative of supercoiled plasmid DNA, and, based on the comparison, determining size and/or a proportion of supercoiled plasmid DNA and/or one or more contaminants, in the fluid sample.

The detected amount of time and/or strength of signal is compared to a reference standard representative of linear plasmid DNA and, based on the comparison, determining size and/or a proportion of linear plasmid DNA and/or one or more contaminants, in the fluid sample.

According to aspects of the present disclosure, the detected amount of time taken by the labeled plasmid DNA of the sample to flow through the polymeric separation medium in the microchannel into the detection region, is compared with the detected amount of time taken by the labeled supercoiled pDNA in the reference standard to flow through the polymeric separation medium in the microchannel into the detection region and/or the detected amount of time taken by the labeled linear pDNA in the reference standard to flow through the polymeric separation medium in the microchannel into the detection region. Based on the comparison, the size and/or conformation of the labeled plasmid DNA of the sample is determined.

According to aspects of the present disclosure, the detected strength of signal of the labeled plasmid DNA is compared with the detected strength of signal of the labeled supercoiled pDNA in the reference standard and/or the detected strength of signal of the labeled linear pDNA in the reference standard. Based on the comparison, amount of the labeled plasmid DNA of the sample is determined.

One or more reference standards may be analyzed at the same time as the fluid sample, e.g. added to the fluid sample, or may be analyzed in parallel, e.g. under similar or the same analysis conditions.

The term “plasmid DNA” as used herein refers to a double-stranded DNA cloning vector used to transfer DNA, such as to transfer DNA from one cell type to another, for example to express an encoded peptide or protein. Plasmid DNA can be used to treat diseases, such as use in gene therapy. Plasmid DNA can be used to prevent or inhibit diseases, such as in vaccines.

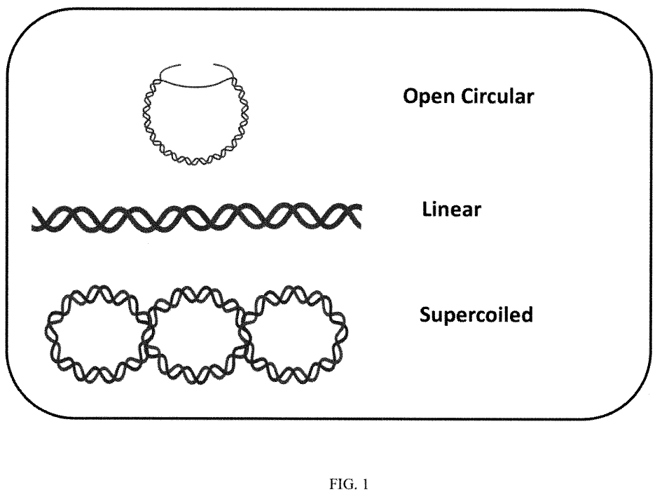

The term “double-stranded plasmid DNA” as used herein refers to plasmid DNA isoforms including 1) linear plasmid DNA and 2) supercoiled plasmid DNA. These isoforms are schematically illustrated in FIG. 1. As used herein, the term “double-stranded plasmid DNA” may include linear plasmid DNA and supercoiled plasmid DNA, Open circle plasmid DNA is considered a degradation isoform impurity which includes small portion(s) of single-stranded DNA. Typically less than 10% of the total length of the double-stranded plasmid DNA molecule is single-stranded, such as less than about 10%, less than about 5%, less than about 1%, less than about 0.1%, less than about 0.01%, less than about 0.001%, or less than about 0.0001%.

The term “contaminant” as used herein refers to DNA other than the desired supercoiled pDNA or desired linear pDNA, such as a multimeric species of the desired supercoiled pDNA or desired linear pDNA, and/or a degraded fragment of the desired supercoiled pDNA or desired linear pDNA such as open circular pDNA.

The term “detectable label” refers to a material capable of producing a signal indicative of the presence of a labeled nucleic acid and detectable by any appropriate method illustratively including spectroscopic, optical, photochemical, biochemical, enzymatic, electrical and/or immunochemical. A detectable label allows for detection based on detectable properties of the label, such as, but not limited to, chemical properties, electrical properties, magnetic properties, optical properties, physical properties, or any two or more thereof. The detectable label may include one or more of: a fluorescent label, a bioluminescent label, a chemiluminescent label, a chromophore, a magnetic label, an antibody, an antigen, an enzyme, a substrate, a radioisotope, or any two or more thereof.

According to aspects of the present disclosure, the detectable label is a fluorescent label. A fluorescent label is selected based on fluorophore characteristics including, but not limited to, excitation maximum wavelength and emission maximum wavelength.

Fluorophores used as fluorescent labels can be any of numerous fluorophores including, but not limited to, those described in Haughland, R. P., The Handbook, A Guide to Fluorescent Probes and Labeling Technologies, 10th Ed., 2005; Lakowicz, J. R., Principles of Fluorescence Spectroscopy, Springer, 3rd ed., 2006; 4-acetamido-4′-isothiocyanatostilbene-2,2′disulfonic acid; acridine and derivatives such as acridine and acridine isothiocyanate; 4-amino-N-[3-vinylsulfonyl)phenyl]naphthalimide-3,5 disulfonate, Lucifer Yellow VS; N-(4-anilino-1-naphthyl) maleimide; anthranilamide, Brilliant Yellow; BIODIPY fluorophores (4,4-difluoro-4-bora-3a,4a-diaza-s-indacenes); coumarin and derivatives such as coumarin, 7-amino-4-methylcoumarin (AMC, Coumarin 120), 7-amino-4-trifluoromethylcouluarin (Coumaran 151);

cyanosine; DAPOXYL sulfonyl chloride; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylaminolnaphthalene-1-sulfonyl chloride (DNS, dansyl chloride); 4-4′-dimethylaminophenylazo)benzoic acid (DABCYL); 4-dimethylaminophenylazophenyl-4′-isothiocyanate (DABITC); EDANS (5-[(2-aminoethyl)amino]naphthalene-1-sulfonic acid), eosin and derivatives such as eosin isothiocyanate; erythrosin and derivatives such as erythrosin B and erythrosin isothiocyanate; ethidium such as ethidium bromide; fluorescein and derivatives such as 5-carboxyfluorescein (FAM), hexachlorofluorescenin, 5-(4,6-dichlorotriazin-2-yl)aminofluorescein (DTAF), 2′, 7′-dimethoxy-4′, 5′-dichloro-6-carboxyfluorescein (JOE) and fluorescein isothiocyanate (FITC); fluorescamine; green fluorescent protein and derivatives such as EBFP, EBFP2, ECFP, and YFP; IAEDANS (5-({2-[(iodoacetyl)amino]ethyl}amino) naphthalene-1-sulfonic acid), Malachite Green isothiocyanate; 4-methylumbelliferone; orthocresolphthalein; nitrotyrosine; pararosaniline; Phenol Red; B-phycoerytnin; o-phthaldialdehyde; pyrene and derivatives such as pyrene butyrate, 1-pyrenesulfonyl chloride and succinimidyl 1-pyrene butyrate; QSY 7; QSY 9; Reactive Red 4 (Cibacron® Brilliant Red 3B-A); rhodamine and derivatives such as 6-carboxy-X-rhodamine (ROX), 6-carboxyrhodamine (Rhodamine 6G), rhodamine isothiocyanate, lissamine rhodamine B sulfonyl chloride, rhodamine B, rhodamine 123, sulforhodamine B, sulforhodamine 101 and sulfonyl chloride derivative of sulforhodamine 101 (Texas Red); N,N,N′,N-tetramethyl-carboxyrhodamine (TAMRA); tetramethyl rhodamine; tetramethyl rhodamine isothiocyanate (TRITC); riboflavin; rosolic acid and terbium chelate derivatives, or any combination of two or more thereof.

According to aspects of the present disclosure, the detectable label is a fluorescent nucleic acid stain. Fluorescent nucleic acid stains include, but are not limited to, Alexa Fluor dyes, BIODIPY dyes, acridine dyes, acridine orange (AO, N,N,N′,N′-Tetramethylacridine-3,6-diamine), cyanine dimer dyes, cyanine monomer dyes, DAPI (4′,6-diamidino-2-phenylindole), DRAQ dyes, ellipticine, ethidium compounds, ethidium bromide, crystal violet, GelRed™, GelGreen™, Hoechst dyes, iodine compounds, 7-aminoactinomycin D, methylene blue, oxazole dyes, PicoGreen, proflavine, propidium iodide (2, 7-Diamino-9-phenyl-10 (diethylaminopropyl)-phenanthridium iodide methiodide), SYBR dyes, SYTO dyes, TOTO™ dyes, thiozole dyes, thiazole orange homodimer (TOTO™-1), thiozole red (TO-PRO®-3), thiazole red homodimer (TOTO®-3), oxazole yellow (YO-PRO®-1), oxazole yellow homodimer (YOYO™-1), oxazole red (YO-PRO®-3), oxazole red homodimer (YOYO™-3), oxazole blue (PO-PRO™-1), oxazole blue homodimer (POPO™-1), TO Iodide (TO-PRO™-1), BOBO, JOJO, LOLO, At BO-PRO, JO-PRO, LO-PRO, or any combination of two or more thereof.

According to preferred aspects of the present disclosure, the detectable label is a fluorescent intercalator. The term “intercalator” refers to a moiety that has higher fluorescence emission when bound to DNA compared to when not bound to DNA and which can insert into an intramolecular space of a DNA molecule, such as between stacked bases of DNA or between stacked base pairs of DNA, forming a fluorescent complex that is stable under conditions of microfluidic electrophoresis such as described herein.

Fluorescent intercalators include, but are not limited to, SYBR dyes, SYTO dyes, ellipticine, ethidium bromide, crystal violet, acridine dyes, acridine orange (AO, N,N,N′,N′-Tetramethylacridine-3,6-diamine), methylene blue, propidium iodide (2, 7-Diamino-9-phenyl-10 (diethylaminopropyl)-phenanthridium iodide methiodide), pyronin Y (Ammonium, (6-(dimethylamino)-3H-xanten-3-ylidene)dimethyl-, chloride, also known as pyronine G, CAS #92-32-0), RiboGreen, RiboRed, 7-aminoactinomycin D, DAPI (4′,6-diamidino-2-phenylindole), TOTO™ dyes, thiozole dyes, thiazole orange homodimer (TOTO™-1), thiozole red (TO-PRO®-3), thiazole red homodimer (TOTO®-3), cyanine dimer dyes, cyanine monomer dyes, proflavine, Alexa Fluor dyes, and BIODIPY dyes.

For labeling the plasmid DNA, the concentration of the label in the sample, aliquot of the sample, or in the polymeric separation medium is about 0.01 mM to about 0.2 mM, such as about 0.025 mM to about 0.15 mM, about 0.05 mM to about 0.1 mM, about 0.075 mM to about 0.1 mM, about 0.01 mM to about 0.025 mM, about 0.01 mM to about 0.05 mM, about 0.01 mM to about 0.075 mM, or about 0.01 mM to about 0.1 mM.

### Device

Methods of assessing a proportion of supercoiled plasmid DNA compared to linear plasmid DNA, and/or impurities such as open circle plasmid DNA and multimers of plasmid DNA in a fluid sample containing plasmid DNA according to aspects of the present disclosure include use of a microfluidic device.

Methods of assessing a proportion of supercoiled plasmid DNA compared to linear plasmid DNA, and/or impurities such as open circle plasmid DNA and multimers of plasmid DNA in a fluid sample containing plasmid DNA according to aspects of the present disclosure include flowing the labeled plasmid DNA through a polymeric separation medium in a microchannel of a microfluidic device.

A microfluidic device used according to aspects of methods of the present disclosure allows for separation of plasmid DNA including supercoiled plasmid DNA, linear plasmid DNA, and impurities such as open circle plasmid DNA and multimers of plasmid DNA when two or more of these are present in a fluid sample and then detection of the separated plasmid DNA types.

According to aspects of the present disclosure, the microfluidic device is a capillary electrophoresis system that allows for separation of supercoiled plasmid DNA, linear plasmid DNA, and impurities such as open circle plasmid DNA and multimers of plasmid DNA when two or more of these are present in a fluid sample. According to aspects of the present disclosure, the microfluidic device is, or includes, a microfluidic chip.

A microfluidic device used according to aspects of methods of the present disclosure includes at least one microchannel and may include one or more receptacles in fluid communication with one or more microchannels. The microchannel(s) and/or receptacle(s) are included in the microfluidic device, for example, by etching, bonding, soft lithography, or molding into a material which is substantially insert with respect to the sieving matrix, dyes, nucleic acid and/or proteins, ceramics and semiconductors, such as glass or silicon, or a polymer, such as polydimethylsiloxane (PDMS). Some or all of the microchannel(s) and/or receptacle(s) may be connected in a network as desired and the microchannel(s) and/or receptacle(s) may be in fluid communication with one or more inputs and/or outputs to allow for input and/or output to/from the microfluidic device.

A microchannel of a microfluidic device is made of any material suitable for containing a polymeric separation medium and aliquot of sample while remaining inert to the polymeric separation medium and aliquot of sample, such as, but not limited to, glass, silicon, plastic, quartz, or mixtures of any two or more thereof.

In some aspects, the microchannel has a length in the range of about 25 millimeters to about 250 millimeters, such as about 25 millimeters to about 35 millimeters, about 35 millimeters to about 45 millimeters, about 45 millimeters to about 55 millimeters, about 55 millimeters to about 65 millimeters, about 65 millimeters to about 75 millimeters, about 75 millimeters to about 85 millimeters, about 85 millimeters to about 95 millimeters, about 95 millimeters to about 100 millimeters, about 100 millimeters to about 110 millimeters, about 110 millimeters to about 120 millimeters, about 120 millimeters to about 130 millimeters, about 130 millimeters to about 140 millimeters, about 140 millimeters to about 150 millimeters, about 150 millimeters to about 160 millimeters, about 160 millimeters to about 170 millimeters, about 170 millimeters to about 180 millimeters, about 180 millimeters to about 190 millimeters, about 190 millimeters to about 200 millimeters, about 200 millimeters to about 210 millimeters, about 210 millimeters to about 220 millimeters, about 220 millimeters to about 230 millimeters, about 230 millimeters to about 240 millimeters, or about 240 millimeters to about 250 millimeters.

In some aspects, the microchannel has an internal diameter of between about 1 micron to about 10 millimeters, such as about 1 micron to about 10 microns, about 10 microns to about 20 microns, about 20 microns to about 30 microns, about 30 microns to about 40 microns, about 40 microns to about 50 microns, about 50 microns to about 60 microns, about 60 microns to about 70 microns, about 70 microns to about 80 microns, about 80 microns to about 90 microns, about 90 microns to about 100 microns, about 100 microns to about 200 microns, about 200 microns to about 300 microns, about 300 microns to about 400 microns, about 400 microns to about 500 microns, about 500 microns to about 600 microns, about 600 microns to about 700 microns, about 700 microns to about 800 microns, about 800 microns to about 900 microns, about 900 microns to about 1 millimeter, or about 1 millimeter to about 10 millimeters.

### Polymeric Separation Medium

According to aspects of the present disclosure an included polymeric separation medium is configured to separate different forms of detectably labeled plasmid DNA, i.e. supercoiled plasmid DNA and linear plasmid DNA, which are, or may be, present in the sample and to obtain a signal from each of the different forms of detectably labeled plasmid DNA, when present, representative of the amount of the different plasmid DNA isoforms of the plasmid DNA present in the sample.

Configuring the polymeric separation medium to separate different forms of detectably labeled plasmid DNA may include, but is not limited to, selection of one or more of: pore size, polymer type, average polymer molecular weight, polymer concentration, cross-linker, extent of polymer cross-linkage, a denaturing agent, a salt, and buffer type.

According to aspects of the present disclosure, the polymeric separation medium, also known as a sieving matrix, is, or includes, a polymer such as, but not limited to, one or more polyacrylamides, polyvinylpyrrolidinones, celluloses, agaroses, or a mixture of any two or more thereof. Polyacrylamides that can be included in the polymeric separation medium include, but are not limited to, linear polyacrylamide, polydimethylacrylamide, polydiethylacrylamide, or a mixture of any two or more thereof. According to aspects of the present disclosure, the polymeric separation medium includes a cellulose polymer, such as, but not limited to, cellulose, hydroxypropyl methylcellulose (HPMC); hydroxyethyl cellulose (HEC); or a mixture of any two or more thereof. According to aspects of the present disclosure, the polymeric separation medium is, or includes, a polymeric gel such as, but not limited to, an acrylamide gel, an agarose gel, or a mixture of any two or more thereof.

The concentration of a polymer in a polymeric separation medium and the viscosity of the polymeric separation medium is adjusted according to the size of the plasmid DNA to be detected. According to aspects of the present disclosure, and included polymeric separation medium has a viscosity in the range of about 0.1 centistokes (cSt) to about 10 cSt. According to aspects of the present disclosure, and included polymeric separation medium has a viscosity in the range of about 0.25 cSt to about 1 cSt.

According to aspects of the present disclosure, the polymeric separation medium includes one or more of: hydroxypropyl methylcellulose (HPMC); hydroxyethyl cellulose (HEC); polyvinylpyrrolidone (PVP); polydimethylacrylamide (PDMA); polyacrylamide gel; and non-cross-linked polyacrylamide.

According to aspects of the present disclosure, a polymer included in the polymeric separation medium is, or includes, a polymer such as, but not limited to, one or more polyvinylpyrrolidinones, celluloses, agaroses, or a mixture of any two or more thereof which is present in a concentration of about 0.08% to about 0.60% weight/weight (w/w) of the total weight of the polymeric separation medium. According to aspects of the present disclosure, a polymer included in the polymeric separation medium is present in a concentration of about 0.08%, about 0.09%, about 0.10%, about 0.11%, about 0.12%, about 0.13%, about 0.14%, about 0.15%, about 0.16%, about 0.17%, about 0.18%, about 0.19%, about 0.20%, about 0.21%, about 0.22%, about 0.23%, about 0.24%, about 0.25%, about 0.26%, about 0.27%, about 0.28%, about 0.29%, about 0.30%, about 0.31%, about 0.32%, about 0.33%, about 0.34%, about 0.35%, about 0.36%, about 0.37%, about 0.38%, about 0.39%, about 0.40%, about 0.41%, about 0.42%, about 0.43%, about 0.44%, about 0.45%, about 0.46%, about 0.47%, about 0.48%, about 0.49%, about 0.50%, about 0.51%, about 0.52%, about 0.53%, about 0.54%, about 0.55%, about 0.56%, about 0.57%, about 0.58%, about 0.59%, or about 0.60% weight/weight (w/w) of the total weight of the polymeric separation medium.

According to aspects of the present disclosure, a polymer included in the polymeric separation medium is, or includes, a cross-linked polyacrylamide, a non-cross-linked polyacrylamide, or a mixture of any two or more thereof which is present in a concentration of 0.05% to 10% weight/weight (w/w) of the total weight of the polymeric separation medium. According to aspects of the present disclosure, a polymer included in the polymeric separation medium is, or includes, a cross-linked polyacrylamide, a non-cross-linked polyacrylamide, or a mixture of any two or more thereof which is present in a concentration of about 0.05%, about 0.10%, about 0.20%, about 0.30%, about 0.40%, about 0.50%, about 0.60%, about 0.70%, about 0.80%, about 0.90%, about 1%, about 2%, about 3%, about 4%, about 5%, about 6%, about 7%, about 8%, about 9%, or about 10% weight/weight (w/w) of the total weight of the polymeric separation medium.

According to aspects of the present disclosure, a polymer included in the polymeric separation medium is, or includes about 5% polyacrylamide.

According to aspects of the present disclosure, the polymeric separation medium includes hydroxypropyl methylcellulose (HPMC) having an average molecular weight in the range of about 80 kDa to about 160 kDa, such as about 80 kDa to about 120 kDa, about 90 kDa to about 160 kDa, about 90 kDa to about 130 kDa, about 100 kDa to about 140 kDa, about 80 kDa to about 110 kDa, about 90 kDa to about 120 kDa, about 100 kDa to about 130 kDa, about 110 kDa to about 140 kDa, about 120 kDa to about 150 kDa, about 130 kDa to about 160 kDa, about 80 kDa to about 100 kDa, about 100 kDa to about 120 kDa, about 120 kDa to about 140 kDa, about 140 kDa to about 160 kDa, about 80 kDa to about 85 kDa, about 85 kDa to about 90 kDa, about 90 kDa to about 95 kDa, about 95 kDa to about 100 kDa, about 100 kDa to about 105 kDa, about 105 kDa to about 110 kDa, about 110 kDa to about 115 kDa, about 115 kDa to about 120 kDa, about 120 kDa to about 125 kDa, about 125 kDa to about 130 kDa, about 130 kDa to about 135 kDa, about 135 kDa to about 140 kDa, about 140 kDa to about 145 kDa, about 145 kDa to about 150 kDa, about 150 kDa to about 155 kDa, or about 155 kDa to about 160 kDa, and present in a concentration of 0.08% to 0.0.6% weight/weight (w/w) of the total weight of the polymeric separation medium.

According to aspects of the present disclosure, the polymeric separation medium includes HPMC having an average molecular weight in the range of about 80 to about 120 kDa and present in a concentration of about 0.08% to about 0.60% weight/weight (w/w) of the total weight of the polymeric separation medium. According to aspects of the present disclosure, the polymeric separation medium includes HPMC having an average molecular weight in the range of about 90 to about 160 kDa and present in a concentration of about 0.08% to about 0.40% weight/weight (w/w) of the total weight of the Polymeric Separation Medium.

According to aspects of the present disclosure, the polymeric separation medium includes hydroxyethyl cellulose (HEC) having an average molecular weight in the range of about 90 to about 160 kDa and present in a concentration of about 0.08% to about 0.60% weight/weight (w/w) of the total weight of the polymeric separation medium.

According to aspects of the present disclosure, the polymeric separation medium includes polyvinylpyrrolidone (PVP) having an average molecular weight in the range of 90-130 kDa and present in a concentration of about 0.08% to about 0.60% weight/weight (w/w) of the total weight of the polymeric separation medium.

According to aspects of the present disclosure, the polymeric separation medium includes polydimethylacrylamide (PDMA), having an average molecular weight in the range of about 80 kDa to about 800 kDa and present in a concentration of about 0.05% to about 0.60% weight/weight (w/w) of the total weight of the polymeric separation medium. According to aspects of the present disclosure, the polymeric separation medium includes polydimethylacrylamide (PDMA), having an average molecular weight in the range of about 80 kDa to about 800 kDa such as about 100 kDa to about 200 kDa, about 200 kDa to about 300 kDa, about 300 kDa to about 400 kDa, about 400 kDa to about 500 kDa, about 500 kDa to about 600 kDa, about 600 kDa to about 700 kDa, about 700 kDa to about 800 kDa, about 150 kDa to about 800 kDa, about 200 kDa to about 800 kDa, about 250 kDa to about 800 kDa, about 300 kDa to about 800 kDa, about 350 kDa to about 800 kDa, about 400 kDa to about 800 kDa, about 450 kDa to about 800 kDa, about 500 kDa to about 800 kDa, about 550 kDa to about 800 kDa, about 600 kDa to about 800 kDa, about 650 kDa to about 800 kDa, about 700 kDa to about 800 kDa, about 750 kDa to about 800 kDa.

According to aspects of the present disclosure, the polymeric separation medium includes polydimethylacrylamide (PDMA), having an average molecular weight in the range of about 80 kDa to about 800 kDa and present in a concentration of about 0.05% to about 0.60% weight/weight (w/w) of the total weight of the polymeric separation medium, such as about 0.05% to about 0.60%, about 0.10% to about 0.60%, about 0.15% to about 0.60%, about 0.20% to about 0.60%, about 0.25% to about 0.60%, about 0.30% to about 0.60%, about 0.35% to about 0.60%, about 0.4% to about 0.6%, about 0.45% to about 0.6%, about 0.5% to about 0.6%, or about 0.55% to about 0.6%.

Optionally, a denaturing agent is included in the polymeric separation medium, such as a chaotropic agent, a detergent, or a mixture thereof.

According to aspects of methods of assessing a proportion of supercoiled plasmid DNA compared to linear plasmid DNA, and/or impurities such as open circle plasmid DNA and multimers of plasmid DNA in a fluid sample comprising plasmid DNA in a fluid sample of the present disclosure, a denaturing agent is included in the polymeric separation medium. According to aspects of methods of assessing a proportion of supercoiled plasmid DNA compared to linear plasmid DNA, and/or impurities such as open circle plasmid DNA and multimers of plasmid DNA in a fluid sample comprising plasmid DNA in a fluid sample of the present disclosure, a denaturing agent included in the polymeric separation medium is, or includes, a chaotropic agent, a detergent, or a mixture thereof.

The denaturing agent can be, or include, a chaotropic agent, such as a thiocyanate salt such as guanidinium thiocyanate, sodium thiocyanate, potassium thiocyanate, or any combination of two or more thereof; n-butanol; ethanol; guanidinium chloride; lithium perchlorate; lithium acetate; magnesium chloride; phenol; 2-propanol; sodium dodecyl sulfate; lithium dodecyl sulfate; thiourea; formamide; urea; DMSO or a combination of any two or more thereof.

The denaturing agent can be, or include, a detergent. The detergent can be an anionic, cationic, zwitterionic, or non-ionic detergent, such as Triton X-100; Triton X-114; NP-40; Tween-20; Tween-80; octyl-beta-glucoside; octylthio glucoside; ethyl trimethyl ammonium bromide; sodium dodecyl sulfate (SDS); Brij-35; Brij-58; CHAPS; CHAPSO; or a combination of any two or more thereof.

The polymeric separation medium may be uniform with respect to pore size along the length of a microchannel in which it is disposed. Alternatively, the polymeric separation medium may be disposed in a non-uniform manner in the microchannel, such as in a smooth gradient of pore size; or in two or more blocks of uniform pore size to achieve a “step” gradient.

According to aspects of methods of assessing a proportion of supercoiled plasmid DNA compared to linear plasmid DNA, and/or impurities such as open circle plasmid DNA and multimers of plasmid DNA in a fluid sample comprising plasmid DNA in a fluid sample of the present disclosure, one or more stabilizers is included in the polymeric separation medium.

The term “stabilizer” as used herein refers to a substance which stabilizes pDNA in the polymeric separation medium such that the proportion of supercoiled plasmid DNA compared to linear plasmid DNA, and/or impurities such as open circle plasmid DNA and multimers of plasmid DNA in the fluid sample remains substantially the same while the pDNA is flowing through the polymeric separation medium in a microfluidic device. A stabilizer further aids in efficient electrophoretic separation.

According to aspects of the present disclosure, the one or more stabilizers included in the polymeric separation medium is or includes one or more of stabilizers selected from the following: urea, magnesium chloride (MgCl₂), tris(hydroxymethyl)methylamino]propanesulfonic acid (TAPS), Tris/Borate/EDTA (TBE), and Tris-acetate-EDTA (TAE).

According to aspects of the present disclosure, the one or more stabilizers included in the polymeric separation medium is or includes one or more of stabilizers selected from the following: tris(hydroxymethyl)methylamino]propanesulfonic acid (TAPS), Tris/Borate/EDTA (TBE), and Tris-acetate-EDTA (TAE).

According to aspects of the present disclosure, the one or more stabilizers included in the polymeric separation medium is or includes one or more of: about 1 mM to about 5 mM MgCl₂ (magnesium chloride); about 5 to about 50 mM Tris Borate EDTA Buffer; about 10 to about 20 mM Tris Buffer, about 10 to about 20 mM Tris HCL Buffer, about 5% to about 30% TAPS buffer (N-Tris(hydroxymethyl)methyl-3-aminopropanesulfonic acid), about 5-15% Tris-acetate-EDTA (TAE), about 5-10% Tris/Borate/EDTA (TBE), about 5 to about 50 mM HEPES (2-[4-(2-hydroxy ethyl) piperazin-1-yl], about 0.1M to about 1M Urea, and about 10 to about 50 mM ethane sulfonic acid) with boric acid.

According to aspects of the present disclosure, the one or more stabilizers included in the polymeric separation medium is or includes one or more of: about 5 to about 50 mM Tris Borate EDTA Buffer; about 10 to about 20 mM Tris Buffer, about 10 to about 20 mM Tris HCL Buffer, about 5% to about 30% TAPS buffer (N-Tris(hydroxymethyl)methyl-3-aminopropanesulfonic acid), about 5-15% Tris-acetate-EDTA (TAE), about 5-10% Tris/Borate/EDTA (TBE), about 5 to about 50 mM HEPES (2-[4-(2-hydroxy ethyl) piperazin-1-yl], and about 10 to about 50 mM ethane sulfonic acid) with boric acid.

According to aspects of the present disclosure, no urea is included in the polymeric separation medium.

According to aspects of the present disclosure, no MgCl₂ is included in the polymeric separation medium.

According to aspects of the present disclosure, the polymeric separation medium includes about 0.3% to about 0.6% polymer gel; about 5% to about 20% TAPS buffer (N-Tris(hydroxymethyl)methyl-3-aminopropanesulfonic acid), and about 0.1M to about 1M Urea, has a conductivity of about 1 ms/cm to about 3 ms/cm, and has a viscosity of about 0.1 centistokes (cSt) to about 10 cSt. According to aspects of the present disclosure, and included polymeric separation medium has a viscosity in the range of about 0.25 cSt to about 1 cSt.

According to aspects of the present disclosure, the polymeric separation medium includes about 0.1% to about 0.6% polydimethylacrylamide (PDMA), having an average molecular weight in the range of about 80 kDa to about 800 kDa; about 5% to about 20% TAPS buffer (N-Tris(hydroxymethyl)methyl-3-aminopropanesulfonic acid), has a conductivity of about 1 ms/cm to about 3 ms/cm, and has a viscosity of about 0.1 cSt to 10 cSt.

According to aspects of the present disclosure, the polymeric separation medium includes about 0.1% to about 0.6% polydimethylacrylamide (PDMA), having an average molecular weight in the range of about 80 kDa to about 800 kDa; about 5% to about 20% TAPS buffer (N-Tris(hydroxymethyl)methyl-3-aminopropanesulfonic acid), has a conductivity of about 1 ms/cm to about 3 ms/cm, and has a viscosity in the range of about 0.25 cSt to about 1 cSt.

According to aspects of the present disclosure, the polymeric separation medium includes about 0.35% to about 0.45% polydimethylacrylamide (PDMA), having an average molecular weight in the range of about 350 kDa to about 450 kDa; about 14% to about 16% TAPS buffer (N-Tris(hydroxymethyl)methyl-3-aminopropanesulfonic acid), has a conductivity of about 2 ms/cm to about 3 ms/cm, and has a viscosity in the range of about 0.3 ml/g to about 0.5 ml/g.

### Flowing

Typically, flowing the labeled plasmid DNA through the polymeric separation medium in the microchannel is performed at a temperature in the range of about 4° C. to about 30° C. According to aspects of the present disclosure, flowing the labeled plasmid DNA through the polymeric separation medium in the microchannel is performed at a temperature in the range of about 4° C. to about 10° C., 10° C. to about 15° C., 15° C. to about 20° C., 20° C. to about 25° C., 25° C. to about 30° C., or about 30° C. to about 40° C.

Typically, flowing the labeled plasmid DNA through the polymeric separation medium in the microchannel includes flow into a detection region in fluid communication with the microchannel, the detection region in signal communication with a sensor capable of detecting a signal from the detectable label of the labeled plasmid DNA.

The labeled plasmid DNA is flowed through the polymeric separation medium to separate different isoforms of labeled plasmid DNA, i.e. supercoiled and linear, as well as contaminants such as open circular pDNA and/or multimers of the pDNA. Flow through the polymeric separation medium from one position in the microchannel towards a distant position in the microchannel is achieved by capillary action, diffusion, hydrodynamic action, and/or promoted by application of pressure gradient, voltage gradient, or a combination of two or more thereof.

Where the microchannel is approximately columnar, flow through the polymeric separation medium from one end of the microchannel towards a distant opposed end of the microchannel is achieved by capillary action, diffusion, hydrodynamic action, and/or promoted by application of pressure gradient, voltage gradient, current gradient, or a combination of two or more thereof along the length of the microchannel.

According to aspects of the present disclosure, a voltage gradient or current gradient is applied to promote flow through the polymeric separation medium from one end of the microchannel towards a distant opposed end of the microchannel. According to aspects of the present disclosure, the applied voltage is in the range of about 500V to about 1500V, such as about 600V to about 1400V, such as about 700V to about 1300V, about 800V to about 1200V, about 900V to about 1100V, or about 1000V. According to aspects of the present disclosure, the applied voltage is in the range of about 1500V to about 2900V, such as about 1500V to about 1600V, about 1500V to about 1700V, about 1500V to about 1800V, about 1500V to about 1900V, about 1500V to about 2000V, about 1500V to about 2100V, about 1500V to about 2200V, about 1500V to about 2300V, about 1500V to about 2400V, about 1500V to about 2500V, about 1500V to about 2600V, about 1500V to about 2700V, about 1500V to about 2800V, about 1600V to about 2900V, about 1700V to about 2900V, about 1800V to about 2900V, about 1900V to about 2900V, about 2000V to about 2900V, about 2100V to about 2900V, about 2200V to about 2900V, about 2300V to about 2900V, about 2400V to about 2900V, about 2500V to about 2900V, about 2600V to about 2900V, about 2700V to about 2900V, about 2800V to about 2900V, about 1500V to about 2000V, about 2000V to about 2500V, or about 2500V to about 2900V.

According to aspects of the present disclosure, the plasmid DNA or the labeled plasmid DNA is introduced into the well and/or microchannel by application of a stimulus effective to urge the movement of the plasmid DNA or the labeled plasmid DNA into the well and/or microchannel of the microfluidic device.

According to aspects of the present disclosure, the plasmid DNA or the labeled plasmid DNA is introduced into the well and/or microchannel by electrokinetic injection from an application device into a well and/or microchannel of a microfluidic device. Electrokinetic injection includes applying a voltage difference to electrokinetically move the plasmid DNA or labeled plasmid DNA from an application device into a well and/or microchannel of a microfluidic device. According to aspects of the present disclosure, the applied voltage is in the range of about 500V to about 1500V, such as about 600V to about 1400V, such as about 700V to about 1300V, about 800V to about 1200V, about 900V to about 1100V, or about 1000V. According to aspects of the present disclosure, the applied voltage is in the range of about 1200V to about 2900V, such as about 1200V to about 1500V, such as about 1500V to about 1600V, about 1500V to about 1700V, about 1500V to about 1800V, about 1500V to about 1900V, about 1500V to about 2000V, about 1500V to about 2100V, about 1500V to about 2200V, about 1500V to about 2300V, about 1500V to about 2400V, about 1500V to about 2500V, about 1500V to about 2600V, about 1500V to about 2700V, about 1500V to about 2800V, about 1600V to about 2900V, about 1700V to about 2900V, about 1800V to about 2900V, about 1900V to about 2900V, about 2000V to about 2900V, about 2100V to about 2900V, about 2200V to about 2900V, about 2300V to about 2900V, about 2400V to about 2900V, about 2500V to about 2900V, about 2600V to about 2900V, about 2700V to about 2900V, about 2800V to about 2900V, about 1500V to about 2000V, about 2000V to about 2500V, or about 2500V to about 2900V.

According to aspects of the present disclosure, the plasmid DNA or the labeled plasmid DNA is introduced into the well and/or microchannel by application of pressure effective to move the plasmid DNA or the labeled plasmid DNA from an application device into a well and/or microchannel of a microfluidic device.

Following introduction into a well and/or microchannel of a microfluidic device, the labeled plasmid DNA is flowed through the polymeric separation medium in the microchannel into a detection region in fluid communication with the microchannel, the detection region in signal communication with a sensor capable of detecting a signal from the detectable label of the labeled plasmid DNA.

Non-limiting examples of sensors capable of detecting a signal from the detectable label of the labeled plasmid DNA include a charge-coupled device (CCD), electron-multiplying CCD, photomultiplier tube, photosensitive diode, a complementary metal-oxide semiconductor (CMOS), an intensified charge-coupled device (ICCD), and an avalanche photodiode.

According to aspects of the present disclosure, the detected signal could be an absorption or a fluorescent signal emitted from a label as a result of contact with electromagnetic radiation which excites the label.

The detected signal may be measured to determine one or more quantitative aspects of the sample, such as one or more of: a) an amount of time taken by the different types of labeled plasmid DNA, i.e. supercoiled plasmid DNA and linear plasmid DNA as well as contaminants such as open circular pDNA and/or multimers of the pDNA, to flow through the polymeric separation medium in the microchannel into the detection region, indicative of conformation and/or size of the different types of labeled plasmid DNA in the fluid sample, and/or b) strength of the signal of the detectable label in the detection region, representative of the amount of the different types of labeled plasmid DNA present, and indicative of concentration of the different types of plasmid DNA in the fluid sample, i.e. one or more of supercoiled plasmid DNA and linear plasmid DNA as well as contaminants such as open circular pDNA and/or multimers of the pDNA. The detected signal(s) allow for assessment of the proportion of supercoiled plasmid DNA compared to linear plasmid DNA as well as contaminants such as open circular pDNA and/or multimers of the pDNA in a fluid sample containing plasmid DNA.

According to aspects of the present disclosure, the detector is operably connected to a computer which stores and manipulates the detected signal information, for example, to calculate one or more parameters relevant to the different isoforms of the pDNA of plasmid DNA in the fluid sample. According to aspects of the present disclosure, the detector is operably connected to a computer which stores and manipulates the detected signal information, for example, to compare the detected signal information to one or more of: a) a reference standard representative of supercoiled plasmid DNA, and b) a reference standard representative of linear plasmid DNA. Based on the comparison of the detected signal information with an appropriate standard or standards, one or more characteristics of the plasmid DNA in the fluid sample is determined, such as 1) size of one or more different isoforms, i.e. supercoiled and/or linear isoforms of labeled plasmid DNA in the fluid sample is detected, and/or 2) the amount of one or more different isoforms, i.e. supercoiled and/or linear isoforms of labeled plasmid DNA in the fluid sample is detected and/or 3) proportion including, for example, at least one ratio of one isoform of plasmid DNA to another isoform of plasmid DNA in the sample, such as a ratio of supercoiled plasmid DNA to linear plasmid DNA, a ratio of supercoiled plasmid DNA and/or linear plasmid DNA to one or more contaminants such as open circular and/or multimers, and/or 4) size of the plasmid DNA, and/or detection of pDNA impurities in the fluid sample.

According to aspects of the present disclosure, the detector is operably connected to a computer which stores and manipulates the detected signal information, for example, to compare the detected signal information to one or more of: a) a reference standard representative of supercoiled plasmid DNA, and/or b) a reference standard representative of linear plasmid DNA. Based on the comparison of the detected signal information with an appropriate standard or standards, a characteristic of the plasmid DNA in the fluid sample is determined, such as 1) conformation of labeled plasmid DNA, 2) size of one or more different isoforms of labeled plasmid DNA in the fluid sample is detected, and/or 3) the amount of one or more different types of labeled plasmid DNA in the fluid sample is detected and/or 4) at least one ratio of one isoform of plasmid DNA to another isoform of plasmid DNA in the sample, such as a ratio of supercoiled plasmid DNA to linear plasmid DNA, a ratio of supercoiled plasmid DNA and/or linear plasmid DNA to a contaminant such as open circular and/or multimers. The detected signal(s) allow for assessment of one or more of: conformation, size, amount, and proportion of supercoiled plasmid DNA compared to linear plasmid DNA, as well as contaminants such as open circular and/or multimers in a fluid sample containing plasmid DNA.

The term “fluid” as used herein in reference to a sample refers to a liquid, gel, or combination of liquid and gel, that can be flowed through a microchannel. According to aspects of the present disclosure, the fluid is an aqueous fluid. According to aspects of the present disclosure, the fluid is an aqueous buffer such as, but not limited to, a Tris buffer, a Tricine buffer, a citrate buffer, a HEPES buffer, a carbonate buffer, a phosphate buffer, a MOPS buffer, a TAPS buffer, and an acetate buffer. Typically, the pH of the aqueous buffer is in the range of about pH 5.0 to about pH 9. According to aspects of the present disclosure, the pH of the aqueous buffer is about pH 5.0 to about pH 5.5, pH 6.0 to about pH 6.5, about pH 6.5 to about pH 7.0, about pH 7.0 to about pH 7.5, about pH 7.5 to about pH 8.0, about pH 8.0 to about pH 8.3, or about pH 8.3 to about pH 9.

The term “sample” as used herein refers to any material that includes, or may include, plasmid DNA of interest.

Optionally, a sample is purified prior to introduction into a microchannel of a microfluidic device. The term “purified” as used herein refers to reduction of at least some contaminating substances such that the purified sample contains a higher amount of plasmid DNA weight/weight than an unpurified sample. According to aspects of the present disclosure, a sample is purified such that contaminating substances in the sample are reduced by at least about 5% or more, at least 10% or more, at least about 20% or more, at least about 30% or more, at least about 40% or more, at least about 50% or more, at least about 60% or more, at least about 70% or more, at least about 80% or more, at least about 90% or more, or at least about 95% or more.

All of a sample, or a portion thereof, may be analyzed according to methods of the present disclosure. The term “aliquot of the fluid sample” refers to a portion of the fluid sample for analysis. The fluid sample, or aliquot thereof, typically has a volume in the range of about 0.5 microliter to about 50 microliters. According to aspects of the present disclosure, the fluid sample, or aliquot thereof, has a volume in the range of about 0.5 microliter to about 5 microliters, about 1 microliter to about 10 microliters, about 1 microliter to about 20 microliters, about 5 microliters to about 10 microliters, about 5 microliters to about 15 microliters, about 5 microliters to about 20 microliters, about 10 microliters to about 15 microliters, about 15 microliters to about 20 microliters, about 25 microliters to about 30 microliters, about 30 microliters to about 35 microliters, about 35 microliters to about 40 microliters, about 40 microliters to about 45 microliters, or about 45 microliters to about 50 microliters. According to aspects of the present disclosure, the fluid sample, or aliquot thereof, has a volume in the range of about 0.5 microliter, about 1 microliter, about 2 microliters, about 3 microliters, about 4 microliters, about 5 microliters, about 6 microliters, about 7 microliters, about 8 microliters, about 9 microliters, or about 10 microliters.

According to aspects of the present disclosure, the plasmid DNA in the fluid sample is present in a concentration in the range of about 0.02 ng/μl to about 10 ng/μl. According to aspects of the present disclosure, the plasmid DNA in the fluid sample is present in a concentration in the range of about 0.02 ng/μl to about 10 ng/μl, about 0.03 ng/μl to about 10 ng/μl, about 0.04 ng/μl to about 10 ng/μl, about 0.05 ng/μl to about 10 ng/μl, about 0.05 ng/μl to about 10 ng/μl, about 0.06 ng/μl to about 10 ng/μl, about 0.07 ng/μl to about 10 ng/μl, about 0.08 ng/μl to about 10 ng/μl, about 0.09 ng/μl to about 10 ng/μl, about 0.1 ng/μl to about 10 ng/μl, about 0.1 ng/μl to about 15.0 ng/μl, about 0.25 ng/μl to about 15.0 ng/μl, about 0.5 ng/μl to about 15.0 ng/μl, about 1.0 ng/μl to about 15.0 ng/μl, about 2.5 ng/μl to about 15.0 ng/μl, about 5.0 ng/μl to about 15.0 ng/μl, about 10.0 ng/μl to about 15.0 ng/μl, about 12.5 ng/μl to about 15.0 ng/μl, about 0.1 ng/μl to about 10.0 ng/μl, about 0.25 ng/μl to about 10.0 ng/μl, about 0.5 ng/μl to about 2.5 ng/μl, about 1.0 ng/μl to about 10.0 ng/μl, about 2.5 ng/μl to about 5.0 ng/μl, about 5.0 ng/μl to about 10.0 ng/μl.

According to aspects of the present disclosure, the plasmid DNA in the fluid sample is present in a concentration in the range of about 0.02 ng/μl to about 10.0 ng/μl. According to aspects of the present disclosure, the plasmid DNA in the fluid sample is present in a concentration in the range of about 0.02 ng/μl to about 10 ng/μl, about 0.03 ng/μl to about 10 ng/μl, about 0.04 ng/μl to about 10 ng/μl, about 0.05 ng/μl to about 10 ng/μl, about 0.05 ng/μl to about 10 ng/μl, about 0.06 ng/μl to about 10 ng/μl, about 0.07 ng/μl to about 10 ng/μl, about 0.08 ng/μl to about 10 ng/μl, about 0.09 ng/μl to about 10 ng/μl, about 0.1 ng/μl to about 10 ng/μl, about 0.1 ng/μl to about 15.0 ng/μl, about 0.25 ng/μl to about 15.0 ng/μl, about 0.5 ng/μl to about 15.0 ng/μl, about 1.0 ng/μl to about 15.0 ng/μl, about 2.5 ng/μl to about 15.0 ng/μl, about 5.0 ng/μl to about 15.0 ng/μl, about 10.0 ng/μl to about 15.0 ng/μl, about 12.5 ng/μl to about 15.0 ng/μl, about 0.1 ng/μl to about 10.0 ng/μl, about 0.25 ng/μl to about 5.0 ng/μl, about 0.5 ng/μl to about 2.5 ng/μl, about 1.0 ng/μl to about 10.0 ng/μl, about 2.5 ng/μl to about 5.0 ng/μl, about 5.0 ng/μl to about 15.0 ng/μl, about 10.0 ng/μl.

According to aspects of the present disclosure, the plasmid DNA in the fluid sample is present in a concentration in the range of about 0.02 ng/μl to about 3.0 ng/μl, about 0.05 ng/μl to about 2.5 ng/μl, about 0.1 ng/μl to about 2 ng/μl, about 0.5 ng/μl to about 2 ng/μl, or about 1 ng/μl to about 2 ng/μl.

According to aspects of the present disclosure, the plasmid DNA in the fluid sample has a size in the range of about 2000 to about 20000 nucleotides in length, such as about 2000 to about 2500 nucleotides in length, about 2500 to about 3000 nucleotides in length, about 3000 to about 3500 nucleotides in length, about 3500 to about 4000 nucleotides in length, about 4000 to about 4500 nucleotides in length, about 4500 to about 5000 nucleotides in length, about 5000 to about 5500 nucleotides in length, about 5500 to about 6000 nucleotides in length, about 6000 to about 6500 nucleotides in length, about 6500 to about 7000 nucleotides in length, about 7000 to about 7500 nucleotides in length, about 8000 to about 8500 nucleotides in length, about 9000 to about 9500 nucleotides in length, about 9500 to about 10000 nucleotides in length, about 11000 to about 11500 nucleotides in length, about 12000 to about 12500 nucleotides in length, about 12500 to about 13000 nucleotides in length, about 13000 to about 13500 nucleotides in length, about 13500 to about 14000 nucleotides in length, about 14000 to about 14500 nucleotides in length, about 14500 to about 15000 nucleotides in length, about 15000 to about 15500 nucleotides in length, about 15500 to about 16000 nucleotides in length, about 16000 to about 16500 nucleotides in length, about 16500 to about 17000 nucleotides in length, about 17000 to about 17500 nucleotides in length, about 17500 to about 18000 nucleotides in length, about 18000 to about 18500 nucleotides in length, about 18500 to about 19000 nucleotides in length, about 19000 to about 19500 nucleotides in length, or about 19500 to about 20000 nucleotides in length.

Embodiments of inventive compositions and methods are illustrated in the following examples. These examples are provided for illustrative purposes and are not considered limitations on the scope of inventive compositions and methods.

### EXAMPLES

The supercoiled samples pBR322 and pTXB1 from New England Biolabs were diluted in TE buffer, pH 7.0 to the specified concentration (˜500 pg/μL). The linear samples were generated using BamHI-HF enzyme, following the protocol provided by the manufacturer, and diluted with TE buffer to the specified concentration. Similarly, the open circular samples were generated using Nb.Bsml enzyme, following the protocol provided by the manufacturer, and then diluted in TE buffer to the specified concentration. Once the samples were generated and combined, they were loaded onto a 96- or 384-well plate and transferred onto the LabChip GXII Touch.

A reference standard including: 1) three supercoiled pDNA fragments having 2.7 kbp, 4.4 kbp, and 6.7 kpb, respectively, in length; and 2) three linear pDNA fragments having 4 kbp, 6 kbp, and 10 kbp, respectively, in length, is dissolved in TE buffer, pH 7.0. Each of the fragments has a concentration of 0.1 ng/μl.

A test sample containing a 4.4 kbp supercoiled pDNA, a 4.4 kbp linear pDNA, and a 4.4 kbp open circle pDNA in TE buffer, pH 7.5 is prepared.

In this example, Ribo Red, or another red nucleic acid dye, is added to a first microtube with the reference standard producing a labeled reference standard and to a second microtube with the test sample producing a labeled test sample.

The polymeric separation medium used in this example includes about 0.4% polydimethylacrylamide (PDMA), having an average molecular weight of about 400 kDa; and about 15% TAPS buffer. The polymeric separation medium has a conductivity of about 2 ms/cm to about 3 ms/cm, and has a viscosity of about 0.3 ml/g to about 0.5 ml/g.

The labeled reference standard is introduced into a first microchannel including the polymeric separation medium by electrokinetic injection. The labeled test sample is introduced into a second microchannel including the polymeric separation medium by electrokinetic injection.

A voltage gradient of about 1000V is applied along the length of the first and second microchannels between the first end and the second end of each microchannel.

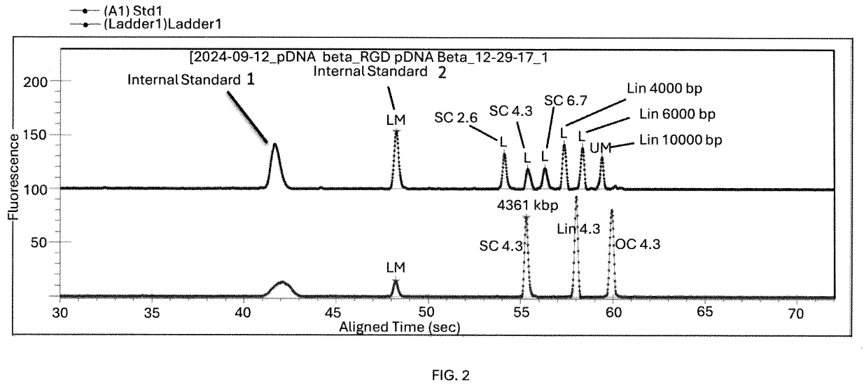

A result similar to that shown in FIG. 2 may be obtained. In FIG. 2, the top line shows the reference standard. A separate peak for each of the three supercoiled pDNA fragments (“SC 2.6”, “SC 4.3”, “SC 6.7”) is observed along with a separate peak for each of the three linear pDNA fragments (“Lin 4000 bp”, “Lin 6000 bp”, “Lin 10000 bp”). The lower line shows three separate peaks for the supercoiled pDNA (“SC-4.3”), linear pDNA (Lin-4.3), and open circular pDNA (OC-4.3) determined by comparison to the reference standard trace above

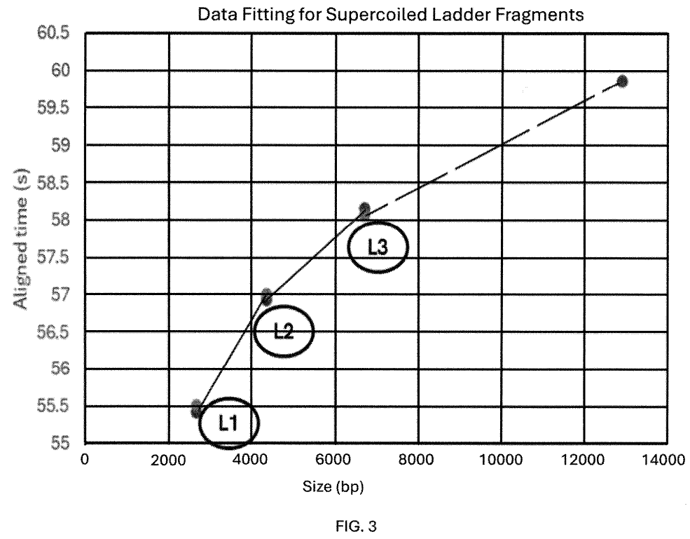

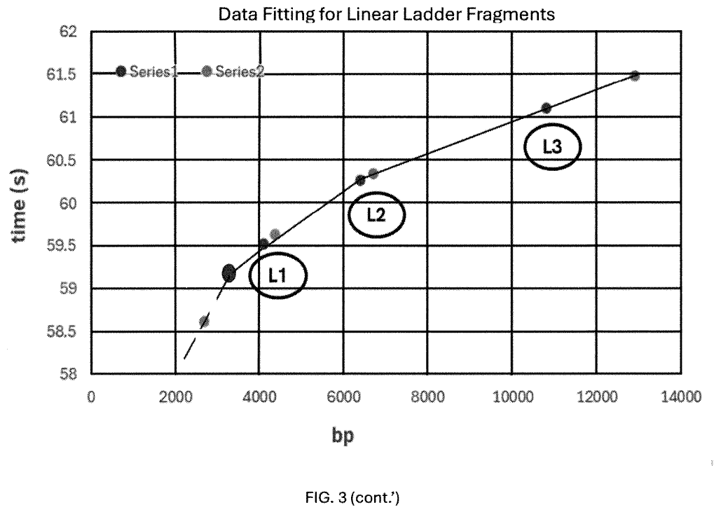

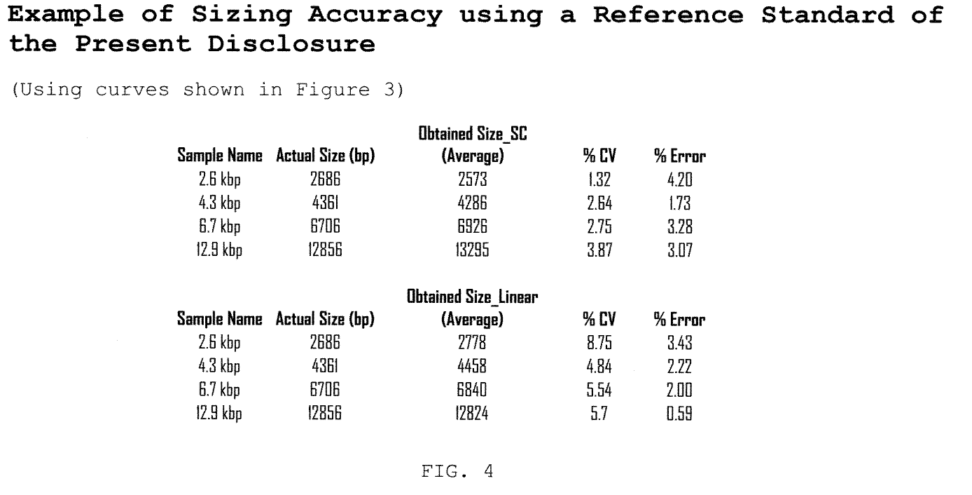

FIG. 3 shows example data of isoform standard curves. using a reference standard of the present disclosure. For each curve, the circled data points L1, L2 and L3 are measured isoform peaks from the ladder. The other data points are measured samples. The solid line represents peak to peak fitting from the reference ladder. The dashed trendline represents predicted sizing experimentally derived from the reference ladder.

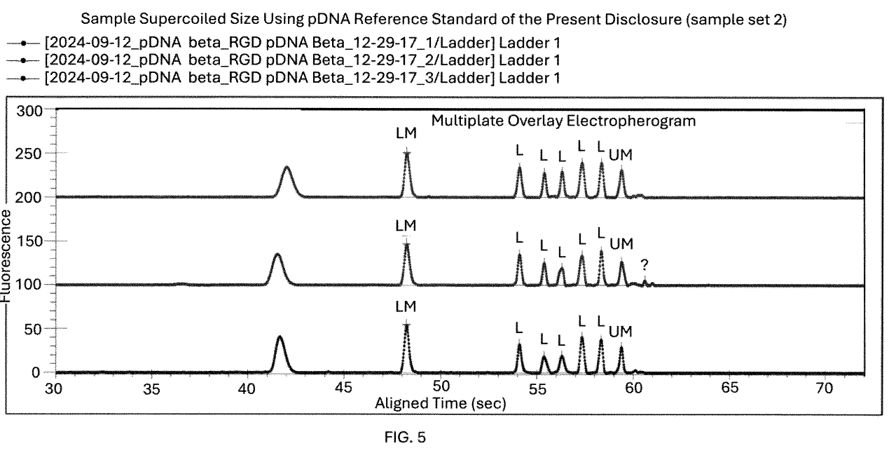

FIG. 4 shows a table listing an example of sizing accuracy using a reference standard of the present disclosure.

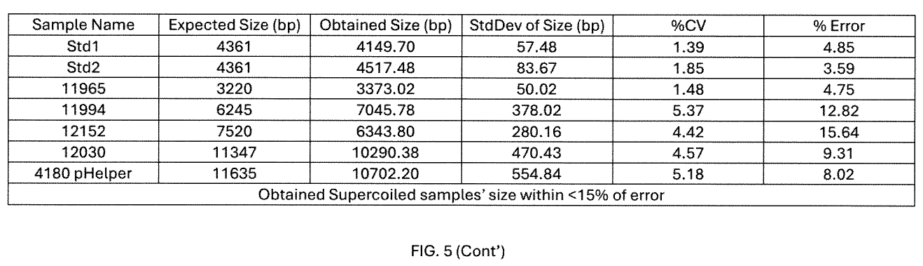

FIG. 5 shows an example of impurity identification using a reference standard of the present disclosure wherein the results were obtained under similar conditions.

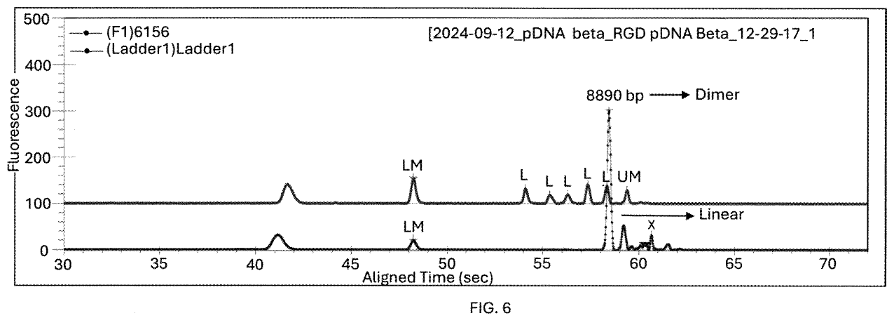

FIG. 6 shows an example of impurity identification using a Reference Standard of the present disclosure. The expected Supercoiled Peak Size: 8890. SC Peak Size Found: 15000 kbp (too large for SC Peak, ˜2× expected size), therefore expected dimer. Second Impurity Peak Found: 8292, Matches sequence size), therefore expected linear peak.

### Item List

Item 1. A method of assessing double-stranded plasmid DNA in a fluid sample to determine one or more of: conformation of the double-stranded plasmid DNA, size of the double-stranded plasmid DNA, and a proportion of supercoiled plasmid DNA compared to linear plasmid DNA, the method comprising: contacting the double-stranded plasmid DNA of the fluid sample with a detectable label which preferentially labels double-stranded plasmid DNA thereby labeling double-stranded plasmid DNA, producing labeled double-stranded plasmid DNA; flowing the labeled double-stranded plasmid DNA through a polymeric separation medium in a microchannel into a detection region in fluid communication with the microchannel, the microchannel having a first end, a second end, and length extending between the first end and the second end, the detection region in signal communication with at least one sensor capable of detecting a signal from the detectable label of the labeled double-stranded plasmid DNA, whereby supercoiled double-stranded plasmid DNA and linear double-stranded plasmid DNA present in the labeled double-stranded plasmid DNA are detectably separated by flowing the labeled double-stranded plasmid DNA through the polymeric separation medium of the microchannel, thereby separating labeled supercoiled plasmid DNA from labeled linear plasmid DNA when both labeled supercoiled plasmid DNA and labeled linear plasmid are present; detecting the detectable label of the labeled double-stranded plasmid DNA in the detection region to determine: a) an amount of time taken by the labeled supercoiled plasmid DNA and/or labeled linear plasmid to flow through the polymeric separation medium in the microchannel into the detection region, indicative of a flow characteristic of the labeled supercoiled plasmid DNA and/or labeled linear plasmid in the fluid sample, and/or b) strength of the signal of the detectable label in the detection region, representative of an amount of supercoiled plasmid DNA and/or linear plasmid DNA, present; and comparing a) and/or b) to a reference standard representative of one or both of: supercoiled plasmid DNA and linear plasmid DNA, wherein the reference standard comprises 1) at least two fragments of supercoiled pDNA of specified sizes and/or 2) at least two fragments of linear pDNA of specified sizes and, based on the comparison, determining one or more of: conformation of the double-stranded plasmid DNA, size of the double-stranded plasmid DNA, and a proportion of supercoiled plasmid DNA compared to linear plasmid DNA in the fluid sample.
Item 2. The method of item 1, wherein the reference standard comprises: 1) at least two fragments of supercoiled pDNA having a size in the range of about 2000 to about 20000 nucleotides in length, wherein the first of the at least two fragments of supercoiled pDNA is smaller than the second of the two fragments of supercoiled pDNA by at least about 1000 nucleotides in length, and/or 2) at least two fragments of linear pDNA in the range of about 2000 to about 20000 nucleotides, wherein the first of the two fragments of linear pDNA is smaller than the second of the two fragments of linear pDNA by at least about 1000 nucleotides in length.
Item 3. The method of item 1, wherein the reference standard comprises 1) at least three fragments of supercoiled pDNA of specified sizes and/or 2) at least three fragments of linear pDNA of specified sizes.
Item 4. The method of item 3, wherein the reference standard comprises: 1) at least three fragments of supercoiled pDNA in the range of about 2000 to about +20000 nucleotides in length, wherein the first of the at least three fragments of supercoiled pDNA is smaller than the second of the at least three fragments of supercoiled pDNA by at least about 1000 nucleotides in length, and the second of the three fragments of supercoiled pDNA is smaller than the third of the at least three fragments by at least about 1000 nucleotides in length; and/or 2) at least three fragments of linear pDNA in the range of about 2000 to about +20000 nucleotides, wherein the first of the three fragments of linear pDNA is smaller than the second of the at least three fragments of linear pDNA by at least about 1000 nucleotides in length, and the second of the at least three fragments of linear pDNA is smaller than the third of the at least three fragments of linear pDNA by at least about 1000 nucleotides in length.
Item 5. The method of item 4, wherein the reference standard comprises: a) a first fragment of supercoiled pDNA having a size in the range of 1.0-3.5 kilobase pairs (kbp), b) a second fragment of supercoiled pDNA having a size in the range of 3.5-6.0 kbp, and c) a third fragment of supercoiled pDNA having a size in the range of 6.0-9.0 kbp, wherein the size of the first fragment of supercoiled pDNA is smaller than the second fragment of supercoiled pDNA by at least 1000 bp, and wherein the size of the second fragment of supercoiled pDNA is smaller than the third fragment of supercoiled pDNA by at least 1000 bp.
Item 6. The method of item 4, wherein the reference standard comprises: d) a first fragment of linear pDNA having a size in the range of 0.2-5.0 kilobase pairs (kbp), e) a second fragment of linear pDNA having a size in the range of 3.0-7.0 kbp, and f) a third fragment of linear pDNA having a size in the range of 5.0-11.0 kbp, wherein the size of the first fragment of linear pDNA is smaller than the second fragment of linear pDNA by at least 1000 bp, and wherein the size of the second fragment of linear pDNA is smaller than the third fragment of linear pDNA by at least 1000 bp.
Item 7. The method of item 4, wherein the reference standard comprises: a) a first fragment of supercoiled pDNA having a size in the range of 1.0-3.5 kilobase pairs (kbp), b) a second fragment of supercoiled pDNA having a size in the range of 2-6.0 kbp, and c) a third fragment of supercoiled pDNA having a size in the range of 6.0-10.0 kbp, wherein the size of the first fragment of supercoiled pDNA is smaller than the second fragment of supercoiled pDNA by at least 1000 bp, and wherein the size of the second fragment of supercoiled pDNA is smaller than the third fragment of supercoiled pDNA by at least 1000 bp; and the reference standard includes d) a first fragment of linear pDNA having a size in the range of 0.2-5.0 kilobase pairs (kbp), e) a second fragment of linear pDNA having a size in the range of 3.0-7.0 kbp, and f) a third fragment of linear pDNA having a size in the range of 5.0-11.0 kbp, wherein the size of the first fragment of linear pDNA is smaller than the second fragment of linear pDNA by at least 1000 bp, and wherein the size of the second fragment of linear pDNA is smaller than the third fragment of linear pDNA by at least 1000 bp.
Item 8. The method of any of items 1 to 7 wherein the concentration of the supercoiled pDNA and/or linear pDNA in the reference standard is in the range of 0.01 ng/μl to about 1 ng/μl for each of the fragments of supercoiled pDNA and/or linear pDNA in the reference standard.
Item 9. The method of any of items 1 to 8, wherein the detectable label is an intercalating agent.
Item 10. The method of any of items 1 to 9, wherein labeling the plasmid DNA comprises introducing the plasmid DNA into a well and/or microchannel of a microfluidic device, the well and/or microchannel comprising the polymeric separation medium and a detectable label which labels plasmid DNA isoforms producing labeled plasmid DNA isoforms in the well and/or microchannel.
Item 11. The method of any of items 1 to 10, wherein the plasmid DNA has a size in the range of about 2000 to about 20000 nucleic acid base pairs in length.
Item 12. The method of any one of items 1 to 11, wherein the plasmid DNA has a concentration in the range of about 0.02 ng/μl to about 2 ng/μl.
Item 13. The method of any one of items 1 to 12, wherein flowing the labeled plasmid DNA through the polymeric separation medium comprises application of a voltage gradient along the length of the microchannel between the first end and the second end, wherein the voltage gradient is in the range of about 500V to about 1500V.
Item 14. The method of any one of items 10 to 13, wherein the labeled plasmid DNA is introduced into the well and/or microchannel by electrokinetic injection or pressure injection.
Item 15. The method of any one of items 1 to 14, wherein the polymeric separation medium comprises a polymer selected from the group consisting of: about 0.08% to about 0.6% hydroxypropyl methylcellulose (HPMC) weight/weight (w/w) of the total weight of the polymeric separation medium having an average molecular weight in the range of about 80 kDa to about 120 kDa; about 0.08% to about 0.4% HPMC, having an average molecular weight in the range of about 90 kDa to about 160 kDa; about 0.08% to about 0.6% weight/weight (w/w) of the total weight of the polymeric separation medium hydroxyethyl cellulose (HEC) having an average molecular weight in the range of about 90 kDa to about 160 kDa; about 0.08% to about 0.6% weight/weight (w/w) of the total weight of the polymeric separation medium polyvinylpyrrolidone (PVP) having an average molecular weight in the range of about 90 kDa to about 130 kDa; about 0.05% to about 0.6% weight/weight (w/w) of the total weight of the polymeric separation medium polydimethylacrylamide (PDMA), having an average molecular weight in the range of about 80 kDa to about 500 kDa; about 5% weight/weight (w/w) of the total weight of the polymeric separation medium polyacrylamide gel; and non-cross-linked polyacrylamide.
Item 16. The method of any one of items 1 to 15, wherein the polymeric separation medium comprises a buffer selected from the group consisting of: 1 mM-5 mM MgCl₂ (magnesium chloride); Tris Borate EDTA Buffer; HEPES (2-[4-(2-hydroxy ethyl) piperazin-1-yl] ethane sulfonic acid) with boric acid; Tris Buffer, and TAPS buffer (N-Tris(hydroxymethyl)methyl-3-aminopropanesulfonic acid).
Item 17. The method of any one of items 1 to 16, wherein the polymeric separation medium comprises a stabilizer.
Item 18. The method of item 17, wherein the stabilizer is selected from the group consisting of: magnesium chloride (MgCl₂), tris(hydroxymethyl)methylamino]propanesulfonic acid (TAPS), Tris/Borate/EDTA (TBE), and Tris-acetate-EDTA (TAE).
Item 19. The method of any one of items 1 to 18, wherein the polymeric separation medium comprises: about 0.3% to about 0.6% polymer gel; and about 5% to about 20% TAPS buffer (N-Tris(hydroxymethyl)methyl-3-aminopropanesulfonic acid), and has a conductivity of about 1 ms/cm to about 3 ms/cm, and has a viscosity of about 0.25 cSt to about 1 cSt.
Item 20. The method of any one of items 1 to 19, wherein the polymeric separation medium comprises: about 0.1% to about 0.6% polydimethylacrylamide (PDMA), having an average molecular weight in the range of about 80 kDa to about 800 kDa; and about 5% to about 20% TAPS buffer (N-Tris(hydroxymethyl)methyl-3-aminopropanesulfonic acid), has a conductivity of about 1 ms/cm to about 3 ms/cm, and has a viscosity of about 0.25 cSt to about 1 cSt.
Item 21. The method of any one of items 1 to 19, wherein a migration time value for a reference standard of a given conformation; and based on alignment of the sample migration time to the standard migration time a sizing output is obtained.
Item 22. A kit for assessing a proportion of supercoiled plasmid DNA compared to linear plasmid DNA, and/or a contaminant, in a fluid sample comprising plasmid DNA, the kit comprising: a DNA reference standard comprising supercoiled pDNA and/or linear pDNA.
Item 23. The kit of item 22, wherein the reference standard includes:
a) a first fragment of supercoiled pDNA having a size in the range of 1.0-3.5 kilobase pairs (kbp), b) a second fragment of supercoiled pDNA having a size in the range of 3.5-6.0 kbp, and c) a third fragment of supercoiled pDNA having a size in the range of 6.0-9.0 kbp, wherein the size of the first fragment of supercoiled pDNA is smaller than the second fragment of supercoiled pDNA by at least 1000 bp, and wherein the size of the second fragment of supercoiled pDNA is smaller than the third fragment of supercoiled pDNA by at least 1000 bp.
Item 24. The kit of item 22 or item 23, wherein the reference standard includes: d) a first fragment of linear pDNA having a size in the range of 0.2-5.0 kilobase pairs (kbp), e) a second fragment of linear pDNA having a size in the range of 3.0-7.0 kbp, and f) a third fragment of linear pDNA having a size in the range of 5.0-11.0 kbp, wherein the size of the first fragment of linear pDNA is smaller than the second fragment of linear pDNA by at least 1000 bp, and wherein the size of the second fragment of linear pDNA is smaller than the third fragment of linear pDNA by at least 1000 bp.
Item 25. The kit of item 22, 23, or 24, wherein the reference standard includes: a) a first fragment of supercoiled pDNA having a size in the range of 1.0-3.5 kilobase pairs (kbp), b) a second fragment of supercoiled pDNA having a size in the range of 2-6.0 kbp, and c) a third fragment of supercoiled pDNA having a size in the range of 6.0-10.0 kbp, wherein the size of the first fragment of supercoiled pDNA is smaller than the second fragment of supercoiled pDNA by at least 1000 bp, and wherein the size of the second fragment of supercoiled pDNA is smaller than the third fragment of supercoiled pDNA by at least 1000 bp; and the reference standard includes d) a first fragment of linear pDNA having a size in the range of 0.2-5.0 kilobase pairs (kbp), e) a second fragment of linear pDNA having a size in the range of 3.0-7.0 kbp, and f) a third fragment of linear pDNA having a size in the range of 5.0-11.0 kbp, wherein the size of the first fragment of linear pDNA is smaller than the second fragment of linear pDNA by at least 1000 bp, and wherein the size of the second fragment of linear pDNA is smaller than the third fragment of linear pDNA by at least 1000 bp.
Item 26. The kit of any of items 22 to 25, further comprising one or more of: a detectable nucleic acid label which labels double-stranded plasmid DNA, a polymeric separation medium, a nucleic acid storage buffer, and a nucleic acid sample buffer.
Item 27. The kit of item 26, wherein the detectable nucleic acid label is fluorescent nucleic acid intercalator.
Item 28. A method of assessing a proportion of supercoiled plasmid DNA compared to linear plasmid DNA, and/or impurities such as open circle plasmid DNA and multimers of plasmid DNA in a fluid sample comprising plasmid DNA substantially as shown or described herein.
Item 29. A kit for assessing a proportion of supercoiled plasmid DNA compared to linear plasmid DNA, and/or impurities such as open circle plasmid DNA and multimers of plasmid DNA in a fluid sample comprising plasmid DNA substantially as shown or described herein.
Item 30. A reference standard substantially as shown or described herein.
Item 31. A DNA reference standard comprising supercoiled pDNA and/or linear pDNA.
Item 32. A DNA reference standard comprising: a) a first fragment of supercoiled pDNA having a size in the range of 1.0-3.5 kilobase pairs (kbp), b) a second fragment of supercoiled pDNA having a size in the range of 3.5-6.0 kbp, and c) a third fragment of supercoiled pDNA having a size in the range of 6.0-9.0 kbp, wherein the size of the first fragment of supercoiled pDNA is smaller than the second fragment of supercoiled pDNA by at least 1000 bp, and wherein the size of the second fragment of supercoiled pDNA is smaller than the third fragment of supercoiled pDNA by at least 1000 bp.
Item 33. The DNA reference standard of item 31 or 32 further comprising: d) a first fragment of linear pDNA having a size in the range of 0.2-5.0 kilobase pairs (kbp), e) a second fragment of linear pDNA having a size in the range of 3.0-7.0 kbp, and f) a third fragment of linear pDNA having a size in the range of 5.0-11.0 kbp, wherein the size of the first fragment of linear pDNA is smaller than the second fragment of linear pDNA by at least 1000 bp, and wherein the size of the second fragment of linear pDNA is smaller than the third fragment of linear pDNA by at least 1000 bp.
Item 34. The DNA reference standard of item 31, 32, or 33, wherein the reference standard includes: a) a first fragment of supercoiled pDNA having a size in the range of 1.0-3.5 kilobase pairs (kbp), b) a second fragment of supercoiled pDNA having a size in the range of 2-6.0 kbp, and c) a third fragment of supercoiled pDNA having a size in the range of 6.0-10.0 kbp, wherein the size of the first fragment of supercoiled pDNA is smaller than the second fragment of supercoiled pDNA by at least 1000 bp, and wherein the size of the second fragment of supercoiled pDNA is smaller than the third fragment of supercoiled pDNA by at least 1000 bp; and the reference standard includes d) a first fragment of linear pDNA having a size in the range of 0.2-5.0 kilobase pairs (kbp), e) a second fragment of linear pDNA having a size in the range of 3.0-7.0 kbp, and f) a third fragment of linear pDNA having a size in the range of 5.0-11.0 kbp, wherein the size of the first fragment of linear pDNA is smaller than the second fragment of linear pDNA by at least 1000 bp, and wherein the size of the second fragment of linear pDNA is smaller than the third fragment of linear pDNA by at least 1000 bp.
Item 35. The DNA reference standard of any of items 31 to 34, further comprising one or more of: a detectable nucleic acid label which labels double-stranded plasmid DNA, a polymeric separation medium, a nucleic acid storage buffer, and a nucleic acid sample buffer.
Item 36. The DNA reference standard of item 35, wherein the detectable nucleic acid label is fluorescent nucleic acid intercalator.

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A method of assessing double-stranded plasmid DNA in a fluid sample to determine one or more of: conformation of the double-stranded plasmid DNA, size of the double-stranded plasmid DNA, and a proportion of supercoiled plasmid DNA compared to linear plasmid DNA, the method comprising:
contacting the double-stranded plasmid DNA of the fluid sample with a detectable label which preferentially labels double-stranded plasmid DNA thereby labeling double-stranded plasmid DNA, producing labeled double-stranded plasmid DNA;
flowing the labeled double-stranded plasmid DNA through a polymeric separation medium in a microchannel into a detection region in fluid communication with the microchannel, the microchannel having a first end, a second end, and length extending between the first end and the second end, the detection region in signal communication with at least one sensor capable of detecting a signal from the detectable label of the labeled double-stranded plasmid DNA, whereby supercoiled double-stranded plasmid DNA and linear double-stranded plasmid DNA present in the labeled double-stranded plasmid DNA are detectably separated by flowing the labeled double-stranded plasmid DNA through the polymeric separation medium of the microchannel, thereby separating labeled supercoiled plasmid DNA from labeled linear plasmid DNA when both labeled supercoiled plasmid DNA and labeled linear plasmid are present;
detecting the detectable label of the labeled double-stranded plasmid DNA in the detection region to determine: a) an amount of time taken by the labeled supercoiled plasmid DNA and/or labeled linear plasmid to flow through the polymeric separation medium in the microchannel into the detection region, indicative of a flow characteristic of the labeled supercoiled plasmid DNA and/or labeled linear plasmid in the fluid sample, and/or b) strength of the signal of the detectable label in the detection region, representative of an amount of supercoiled plasmid DNA and/or linear plasmid DNA, present; and
comparing a) and/or b) to a reference standard representative of one or both of: supercoiled plasmid DNA and linear plasmid DNA, wherein the reference standard comprises 1) at least two fragments of supercoiled pDNA of specified sizes and/or 2) at least two fragments of linear pDNA of specified sizes and, based on the comparison, determining one or more of: conformation of the double-stranded plasmid DNA, size of the double-stranded plasmid DNA, and a proportion of supercoiled plasmid DNA compared to linear plasmid DNA in the fluid sample.

2. The method of claim 1, wherein the reference standard comprises: 1) at least two fragments of supercoiled pDNA having a size in the range of about 2000 to about 20000 nucleotides in length, wherein the first of the at least two fragments of supercoiled pDNA is smaller than the second of the two fragments of supercoiled pDNA by at least about 1000 nucleotides in length, and/or 2) at least two fragments of linear pDNA in the range of about 2000 to about 20000 nucleotides, wherein the first of the two fragments of linear pDNA is smaller than the second of the two fragments of linear pDNA by at least about 1000 nucleotides in length.

3. The method of claim 1, wherein the reference standard comprises 1) at least three fragments of supercoiled pDNA of specified sizes and/or 2) at least three fragments of linear pDNA of specified sizes.

4. The method of claim 3, wherein the reference standard comprises: 1) at least three fragments of supercoiled pDNA in the range of about 2000 to about +20000 nucleotides in length, wherein the first of the at least three fragments of supercoiled pDNA is smaller than the second of the at least three fragments of supercoiled pDNA by at least about 1000 nucleotides in length, and the second of the three fragments of supercoiled pDNA is smaller than the third of the at least three fragments by at least about 1000 nucleotides in length; and/or 2) at least three fragments of linear pDNA in the range of about 2000 to about +20000 nucleotides, wherein the first of the three fragments of linear pDNA is smaller than the second of the at least three fragments of linear pDNA by at least about 1000 nucleotides in length, and the second of the at least three fragments of linear pDNA is smaller than the third of the at least three fragments of linear pDNA by at least about 1000 nucleotides in length.

5. The method of claim 4, wherein the reference standard comprises: a) a first fragment of supercoiled pDNA having a size in the range of 1.0-3.5 kilobase pairs (kbp), b) a second fragment of supercoiled pDNA having a size in the range of 3.5-6.0 kbp, and c) a third fragment of supercoiled pDNA having a size in the range of 6.0-9.0 kbp, wherein the size of the first fragment of supercoiled pDNA is smaller than the second fragment of supercoiled pDNA by at least 1000 bp, and wherein the size of the second fragment of supercoiled pDNA is smaller than the third fragment of supercoiled pDNA by at least 1000 bp.

6. The method of claim 4, wherein the reference standard comprises: d) a first fragment of linear pDNA having a size in the range of 0.2-5.0 kilobase pairs (kbp), e) a second fragment of linear pDNA having a size in the range of 3.0-7.0 kbp, and f) a third fragment of linear pDNA having a size in the range of 5.0-11.0 kbp, wherein the size of the first fragment of linear pDNA is smaller than the second fragment of linear pDNA by at least 1000 bp, and wherein the size of the second fragment of linear pDNA is smaller than the third fragment of linear pDNA by at least 1000 bp.

7. The method of claim 4, wherein the reference standard comprises: a) a first fragment of supercoiled pDNA having a size in the range of 1.0-3.5 kilobase pairs (kbp), b) a second fragment of supercoiled pDNA having a size in the range of 2-6.0 kbp, and c) a third fragment of supercoiled pDNA having a size in the range of 6.0-10.0 kbp, wherein the size of the first fragment of supercoiled pDNA is smaller than the second fragment of supercoiled pDNA by at least 1000 bp, and wherein the size of the second fragment of supercoiled pDNA is smaller than the third fragment of supercoiled pDNA by at least 1000 bp; and the reference standard includes d) a first fragment of linear pDNA having a size in the range of 0.2-5.0 kilobase pairs (kbp), e) a second fragment of linear pDNA having a size in the range of 3.0-7.0 kbp, and f) a third fragment of linear pDNA having a size in the range of 5.0-11.0 kbp, wherein the size of the first fragment of linear pDNA is smaller than the second fragment of linear pDNA by at least 1000 bp, and wherein the size of the second fragment of linear pDNA is smaller than the third fragment of linear pDNA by at least 1000 bp.

8. The method of claim 1, wherein the concentration of the supercoiled pDNA and/or linear pDNA in the reference standard is in the range of 0.01 ng/μl to about 1 ng/μl for each of the fragments of supercoiled pDNA and/or linear pDNA in the reference standard.

9. The method of claim 1, wherein the detectable label is an intercalating agent.

10. The method of claim 1, wherein the plasmid DNA has a size in the range of about 2000 to about 20000 nucleic acid base pairs in length and/or wherein the plasmid DNA has a concentration in the range of about 0.02 ng/μl to about 2 ng/μl.

11. The method of claim 1, wherein flowing the labeled plasmid DNA through the polymeric separation medium comprises application of a voltage gradient along the length of the microchannel between the first end and the second end, wherein the voltage gradient is in the range of about 500V to about 1500V.

12. The method of claim 1, wherein the polymeric separation medium comprises a polymer selected from the group consisting of: about 0.08% to about 0.6% hydroxypropyl methylcellulose (HPMC) weight/weight (w/w) of the total weight of the polymeric separation medium having an average molecular weight in the range of about 80 kDa to about 120 kDa; about 0.08% to about 0.4% HPMC, having an average molecular weight in the range of about 90 kDa to about 160 kDa; about 0.08% to about 0.6% weight/weight (w/w) of the total weight of the polymeric separation medium hydroxyethyl cellulose (HEC) having an average molecular weight in the range of about 90 kDa to about 160 kDa; about 0.08% to about 0.6% weight/weight (w/w) of the total weight of the polymeric separation medium polyvinylpyrrolidone (PVP) having an average molecular weight in the range of about 90 kDa to about 130 kDa; about 0.05% to about 0.6% weight/weight (w/w) of the total weight of the polymeric separation medium polydimethylacrylamide (PDMA), having an average molecular weight in the range of about 80 kDa to about 500 kDa; about 5% weight/weight (w/w) of the total weight of the polymeric separation medium polyacrylamide gel; and non-cross-linked polyacrylamide.

13. The method of claim 1, wherein the polymeric separation medium comprises a buffer selected from the group consisting of: 1 mM-5 mM MgCl₂ (magnesium chloride); Tris Borate EDTA Buffer; HEPES (2-[4-(2-hydroxy ethyl) piperazin-1-yl] ethane sulfonic acid) with boric acid; Tris Buffer, and TAPS buffer (N-Tris(hydroxymethyl)methyl-3-aminopropanesulfonic acid).

14. The method of claim 1, wherein the polymeric separation medium comprises: about 0.3% to about 0.6% polymer gel; and about 5% to about 20% TAPS buffer (N-Tris(hydroxymethyl)methyl-3-aminopropanesulfonic acid), and has a conductivity of about 1 ms/cm to about 3 ms/cm, and has a viscosity of about 0.25 cSt to about 1 cSt.

15. The method of claim 1, wherein the polymeric separation medium comprises: about 0.1% to about 0.6% polydimethylacrylamide (PDMA), having an average molecular weight in the range of about 80 kDa to about 800 kDa; and about 5% to about 20% TAPS buffer (N-Tris(hydroxymethyl)methyl-3-aminopropanesulfonic acid), has a conductivity of about 1 ms/cm to about 3 ms/cm, and has a viscosity of about 0.25 cSt to about 1 cSt.

16. A kit for assessing a proportion of supercoiled plasmid DNA compared to linear plasmid DNA, and/or a contaminant, in a fluid sample comprising plasmid DNA, the kit comprising: a DNA reference standard comprising supercoiled pDNA and/or linear pDNA.

17. The kit of claim 16, wherein the reference standard includes:
a) a first fragment of supercoiled pDNA having a size in the range of 1.0-3.5 kilobase pairs (kbp), b) a second fragment of supercoiled pDNA having a size in the range of 3.5-6.0 kbp, and c) a third fragment of supercoiled pDNA having a size in the range of 6.0-9.0 kbp, wherein the size of the first fragment of supercoiled pDNA is smaller than the second fragment of supercoiled pDNA by at least 1000 bp, and wherein the size of the second fragment of supercoiled pDNA is smaller than the third fragment of supercoiled pDNA by at least 1000 bp.

18. The kit of claim 16, wherein the reference standard includes: d) a first fragment of linear pDNA having a size in the range of 0.2-5.0 kilobase pairs (kbp), e) a second fragment of linear pDNA having a size in the range of 3.0-7.0 kbp, and f) a third fragment of linear pDNA having a size in the range of 5.0-11.0 kbp, wherein the size of the first fragment of linear pDNA is smaller than the second fragment of linear pDNA by at least 1000 bp, and wherein the size of the second fragment of linear pDNA is smaller than the third fragment of linear pDNA by at least 1000 bp.

19. The kit of claim 16, wherein the reference standard includes: a) a first fragment of supercoiled pDNA having a size in the range of 1.0-3.5 kilobase pairs (kbp), b) a second fragment of supercoiled pDNA having a size in the range of 2-6.0 kbp, and c) a third fragment of supercoiled pDNA having a size in the range of 6.0-10.0 kbp, wherein the size of the first fragment of supercoiled pDNA is smaller than the second fragment of supercoiled pDNA by at least 1000 bp, and wherein the size of the second fragment of supercoiled pDNA is smaller than the third fragment of supercoiled pDNA by at least 1000 bp; and the reference standard includes d) a first fragment of linear pDNA having a size in the range of 0.2-5.0 kilobase pairs (kbp), e) a second fragment of linear pDNA having a size in the range of 3.0-7.0 kbp, and f) a third fragment of linear pDNA having a size in the range of 5.0-11.0 kbp, wherein the size of the first fragment of linear pDNA is smaller than the second fragment of linear pDNA by at least 1000 bp, and wherein the size of the second fragment of linear pDNA is smaller than the third fragment of linear pDNA by at least 1000 bp.

20. The kit of claim 16, further comprising one or more of: a detectable nucleic acid label which labels double-stranded plasmid DNA, a polymeric separation medium, a nucleic acid storage buffer, and a nucleic acid sample buffer.
